(19) United States
(12) Patent Application Publication
(10) Pub. No.: US 2026/0114923 A1
(43) Pub. Date: Apr. 30, 2026

(54) ELECTROSURGICAL SYSTEM HAVING POSITIONAL FEEDBACK DURING TISSUE TRANSVERSAL

(71) Applicant: Boston Scientific Scimed, Inc., Maple Grove, MN (US)

(72) Inventors: Steven Kinio, Pickering (CA); Eduardo Moriyama, Richmond (CA); Christian Balkovec, Cambridge (CA); Gareth Davies, Toronto (CA); Deep Patel, Waterdown (CA)

(21) Appl. No.: 19/374,394

(22) Filed: Oct. 30, 2025

Related U.S. Application Data
(60) Provisional application No. 63/714,586, filed on Oct. 31, 2024.

(51) Int. Cl. A61B 18/16; A61B 18/00; A61B 18/14; A61B 90/00

(52) U.S. Cl. CPC A61B 18/16; A61B 18/1492; A61B 90/06; A61B 2018/00351; A61B 2018/00625; A61B 2018/00642; A61B 2018/00666; A61B 2018/00708; A61B 2018/00791; A61B 2090/064

(57) ABSTRACT

An electrosurgical unit for use with a crossing member is disclosed. The electrosurgical unit includes a controller and device terminals. The device terminals include an active terminal and a measurement terminal. The device terminals couple to the crossing member. The controller generates a radiofrequency (RF) energy signal that is delivered to the active terminal. The RF energy signal is configured to cross tissue. The controller also determines whether the crossing member has crossed the tissue via a determined change in a response to a measurement signal received at the measurement terminal and generates an indication of tissue crossing based on the determined change in the response to the measurement signal.

### CROSS REFERENCE TO RELATED APPLICATIONS

This application claims priority to U.S. Provisional Patent Application No. 63/714,586 entitled “ELECTROSURGICAL SYSTEM HAVING POSITIONAL FEEDBACK DURING TISSUE TRANSVERSAL,” filed Oct. 31, 2024, which is incorporated herewith by reference in its entirety.

### TECHNICAL FIELD

The present disclosure relates to medical devices and systems for use in percutaneous or interventional procedures including surgery such as electrophysiology procedures. More specifically, this disclosure relates to electrosurgical units, such as radiofrequency (RF) generators, electrosurgical systems, and methods, that provide for measurements of electrical characteristics from electrosurgical access devices such as crossing members used with RF generators.

### BACKGROUND

Electrosurgery is the application of RF energy to biological tissue to perform several functions including cut, puncture, coagulate, desiccate, or fulgurate the tissue. Electrosurgery is typically performed with electrosurgical systems having an electrosurgical unit such as a generator to provide a source of RF energy coupled to an electrosurgical device having an electrode to apply the RF energy to the biological tissue. In one example, electrosurgical systems can provide access in surgery such as incisions or holes in tissue, or cross tissue, for clinicians to access biological features past the incisions or holes. The present disclosure includes catheters as examples of electrosurgical crossing devices in electrosurgery for illustration, and a transseptal puncture as an example of an electrosurgical crossing, but the systems and procedures described in this disclosure can be applied to other forms of electrosurgical access and electrosurgical devices including devices used to provide incisions in laparoscopic surgery.

Catheters are often used to provide general access into a patient's body using minimally invasive techniques. In some examples, a catheter can be used to create a channel through a region of the body. For instance, punctures in tissues can provide access for medical tools used in various medical interventions. In one example, a pericardium layer of a patient can be punctured to provide for epicardial access, such as to create an access point to insert tools for epicardial ablation. In another example, electrosurgical devices are applied to remove accumulation of atheromatous material on the inner walls of vascular lumens, which results in atherosclerosis. In one technique, an electrosurgical device is applied to puncture through the vascular occlusion without affecting the vessel walls. Another example is a transseptal puncture in a cardiac procedure. The left atrium is a difficult cardiac chamber to reach percutaneously. Although the left atrium can be reached via the left ventricle and mitral valve, the catheter is manipulated through two U-turns, which can be cumbersome. the transseptal puncture is a technique of creating a small passage through the atrial septum, or wall in the heart between the left and right atrium, through which a catheter can be fed. The atrial septum is punctured and dilated via tools. The transseptal puncture permits a direct route to the left atrium via the intra-atrial septum and systematic venous system. Increasing larger and complex medical devices can be passed into the right atrium.

Punctures, such as transseptal punctures, can be performed with the aid of electrosurgical crossing devices, such as transseptal guidewires, having electrodes energized with a suitable power source such as an electrically coupled electrosurgical generator to provide the source of RF energy in a manner like other electrosurgical instruments. Typical electrosurgical instruments including electrosurgical crossing devices apply an electrical potential difference or a voltage difference between an active electrode and a return electrode on a patient's grounded body in a monopolar arrangement of the device or between an active electrode and a return electrode on the device in bipolar arrangement to deliver the RF energy to the area where tissue is to be affected. Electrosurgical crossing devices pass RF energy through tissue between the electrodes to puncture tissue with plasma formed on the energized electrode. Tissue that contacts the plasma experiences a rapid vaporization of cellular fluid to produce a puncture effect. Electrical energy can be applied to the electrodes either as a train of high frequency pulses or as a continuous signal typically in the RF range to perform the puncturing techniques.

### SUMMARY

In Example 1, an electrosurgical unit for use with a crossing member, the electrosurgical unit comprising: a plurality of device terminals including an active terminal and a measurement terminal, the plurality of device terminals configured to couple to the crossing member; and a controller configured to: generate a radiofrequency (RF) energy signal delivery to the active terminal, the RF energy signal configured to cross tissue; determine whether the crossing member has crossed the tissue via a determined change in a response to a measurement signal received at the measurement terminal; and generate an indication of tissue crossing based on the determined change in the response to the measurement signal.

In Example 2, the electrosurgical unit of Example 1, wherein the determined change in the response to the measurement signal is based on a threshold.

In Example 3, the electrosurgical unit of Example 2, wherein the controller is configured to determine a time-of-flight from the response to the measurement signal, and the determined change in the response to the measurement signal is based on a change of the determined time-of-flight with respect to a threshold time-of-flight.

In Example 4, the electrosurgical unit of Example 1, wherein the determined change in the response to the measurement signal is based on a comparison to a stored profile.

In Example 5, the electrosurgical unit of Example 4, wherein the controller is configured to determine a spectroscopic profile from the response to the measurement signal, and the determined change in the response to the measurement signal is based on a change of the determined spectroscopic profile from a comparison to a stored spectroscopic profile.

In Example 6, the electrosurgical unit of any of Examples 1, 2 and 4, and further comprising a measurement circuit coupled to the controller and the measurement terminal to receive the response to the measurement signal.

In Example 7, the electrosurgical unit of Example 6, wherein the measurement circuit generates the measurement signal.

In Example 8, the electrosurgical unit of any of Examples 6 and 7, wherein the measurement circuit is an optical measurement circuit having an optical transducer.

In Example 9, the electrosurgical unit of Example 8, wherein the optical measurement circuit is configured to generate an optical measurement signal provided to the measurement terminal.

In Example 10, the electrosurgical unit of any of Examples 6 and 7, wherein the measurement circuit is a thermal measurement circuit configured to process a signal from a thermal sensor.

In Example 11, the electrosurgical unit of any of Examples 6 and 7, wherein the measurement circuit is a pressure measurement circuit configured to process a signal from a pressure sensor.

In Example 12, the electrosurgical unit of Example 11, wherein the pressure measurement circuit includes a pressure transducer.

In Example 13, the electrosurgical unit of any of Examples 1-12, wherein the controller configured to generate the indication of tissue crossing includes the controller configured to terminate the RF energy signal based on the determined change in the response to the measurement signal.

In Example 14, the electrosurgical unit of any of Examples 1, 2 and 4, wherein the determined change in a response to a measurement signal is based on a determined change in a measurement of electrical permittivity of the tissue.

In Example 15, the electrosurgical unit of any of Examples 1-14, wherein the crossing member further includes a delivery component for accessing a heart.

In Example 16, an electrosurgical unit for use with a crossing member, the electrosurgical unit comprising: a plurality of device terminals including an active terminal and a measurement terminal, the plurality of device terminals configured to couple to the crossing member; and a controller configured to: generate a radiofrequency (RF) energy signal delivery to the active terminal, the RF energy signal configured to cross tissue; determine whether the crossing member has crossed the tissue via a determined change in a response to a measurement signal received at the measurement terminal; and generate an indication of tissue crossing based on the determined change in the response to the measurement signal.

In Example 17, the electrosurgical unit of Example 16, wherein the determined change in the response to the measurement signal is based on a threshold.

In Example 18, the electrosurgical unit of Example 17, wherein the controller is configured to determine a time-of-flight from the response to the measurement signal, and the determined change in the response to the measurement signal is based on a change of the determined time-of-flight with respect to a threshold time-of-flight.

In Example 19, the electrosurgical unit of Example 16, wherein the determined change in the response to the measurement signal is based on a comparison to a stored profile.

In Example 20, the electrosurgical unit of Example 19, wherein the controller is configured to determine a spectroscopic profile from the response to the measurement signal, and the determined change in the response to the measurement signal is based on a change of the determined spectroscopic profile from a comparison to a stored spectroscopic profile.

In Example 21, the electrosurgical unit of Example 16, and further comprising a measurement circuit coupled to the controller and the measurement terminal to receive the response to the measurement signal.

In Example 22, the electrosurgical unit of Example 21, wherein the measurement circuit generates the measurement signal.

In Example 23, the electrosurgical unit of Example 21, wherein the measurement circuit is an optical measurement circuit having an optical transducer.

In Example 24, the electrosurgical unit of Example 23, wherein the optical measurement circuit is configured to generate an optical measurement signal provided to the measurement terminal.

In Example 25, the electrosurgical unit of Example 21, wherein the measurement circuit is a thermal measurement circuit configured to process a signal from a thermal sensor.

In Example 26, the electrosurgical unit of Example 21, wherein the measurement circuit is a pressure measurement circuit configured to process a signal from a pressure sensor.

In Example 27, the electrosurgical unit of Example 26, wherein the pressure measurement circuit includes a pressure transducer.

In Example 28, the electrosurgical unit of Example 16, wherein the controller configured to generate the indication of tissue crossing includes the controller configured to terminate the RF energy signal based on the determined change in the response to the measurement signal.

In Example 29, the electrosurgical unit of Example 16, wherein the determined change in a response to a measurement signal is based on a determined change in a measurement of electrical permittivity of the tissue.

In Example 30, an electrosurgical unit for use with a crossing member, the electrosurgical unit comprising: a plurality of device terminals including an active terminal and a measurement terminal, the plurality of device terminals configured to couple to the crossing member; and a controller configured to: generate a radiofrequency (RF) energy signal delivery to the active terminal, the RF energy signal configured to cross tissue; receive a response to a measurement signal at the measurement terminal; determine whether the crossing member has crossed the tissue via a determined change in the response to the measurement signal; and terminate the RF energy signal based on the determined change in the response to the measurement signal.

In Example 31, the electrosurgical unit of Example 30, wherein the determined change in the response to the measurement signal is based on one of a threshold or a comparison to a stored profile.

In Example 32, the electrosurgical unit of Example 30, and further comprising a measurement circuit coupled to the controller and the measurement terminal to receive the response to the measurement signal.

In Example 33, an electrosurgical tissue crossing system comprising: a plurality of device terminals including an active terminal, a return terminal, and a measurement terminal; a crossing member having distal portion including a crossing electrode and a measurement probe; the crossing electrode electrically coupled to the active terminal, the measurement probe operably coupled to the measurement terminal; a ground pad dispersive electrode electrically coupled to the return terminal; and a controller configured to: generate a radiofrequency (RF) energy signal delivery to the active terminal, the RF energy signal configured to cross tissue; generate a measurement signal, the measurement signal configured to be delivered to the measurement probe; receive a response to the measurement signal at the measurement terminal, the response from the measurement signal received from the measurement probe; determine whether the crossing member has crossed the tissue via a determined change in the response to the measurement signal; and terminate the RF energy signal based on the determined change in the response to the measurement signal.

In Example 34, the electrosurgical tissue crossing system of Example 33, wherein the determined change in a response to a measurement signal is based on a determined change in a measurement of electrical permittivity of the tissue.

In Example 35, the electrosurgical tissue crossing system of Example 33, wherein the crossing member further includes a delivery component for accessing a heart.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic diagram illustrating an exemplary electrosurgical system for performing an electrosurgical puncture, such as a transseptal puncture.

FIG. 2 is a schematic diagram illustrating an embodiment of an electrosurgical generator for use in the electrosurgical system of FIG. 1.

FIG. 3 is a flow diagram illustrating an example process of the electrosurgical generator of FIG. 2.

FIG. 4 is a schematic diagram illustrating an embodiment of the electrosurgical system of FIG. 1.

FIG. 5 is a flow diagram illustrating an embodiment of the example process illustrated in FIG. 3 implemented with an embodiment of the electrosurgical system illustrated in FIG. 4.

FIG. 6 is a schematic diagram illustrating another embodiment of an electrosurgical generator for use in the electrosurgical system of FIG. 1.

FIG. 7 is a schematic diagram illustrating another embodiment of the electrosurgical system of FIG. 1.

FIG. 8 is a flow diagram illustrating an embodiment of the example process illustrated in FIG. 3 implemented with an embodiment of the electrosurgical system illustrated in FIG. 7.

FIG. 9 is a flow diagram illustrating another embodiment of the example process illustrated in FIG. 3 implemented with an embodiment of the electrosurgical system illustrated in FIG. 7.

FIG. 10 is a schematic diagram illustrating another embodiment of the electrosurgical system of FIG. 1.

FIG. 11 is a flow diagram illustrating an embodiment of the example process illustrated in FIG. 3 implemented with an embodiment of the electrosurgical system illustrated in FIG. 10.

FIG. 12 is a flow diagram illustrating another embodiment of the example process illustrated in FIG. 3 implemented with an embodiment of the electrosurgical system illustrated in FIG. 10.

FIG. 13 is a schematic diagram illustrating another embodiment of the electrosurgical system of FIG. 1.

FIG. 14 is a flow diagram illustrating an embodiment of the example process illustrated in FIG. 3 implemented with an embodiment of the electrosurgical system illustrated in FIG. 13.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

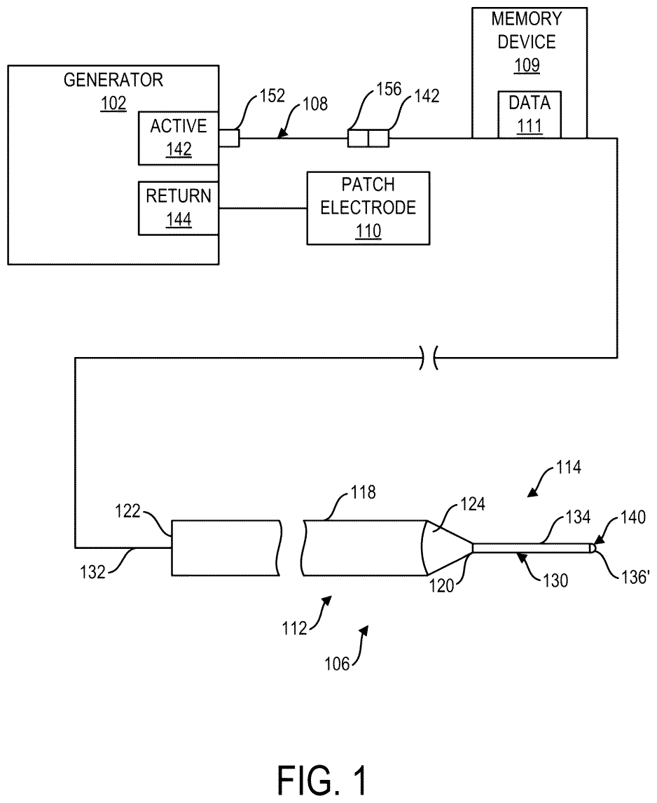

FIG. 1 illustrates an embodiment of an electrosurgical system 100 to cut, such as cross, biological tissue of a patient. More particularly, the electrosurgical system of the illustrated embodiment facilitates vascular access to a heart and provides catheter positioning within cardiac anatomy. The embodiment of the electrosurgical system 100 includes an electrosurgical generator 102 and an electrosurgical crossing assembly 106. The electrosurgical generator 102 is an embodiment of an electrosurgical units. The electrosurgical system 100 can include additional electrosurgical units such as an electroanatomical mapping system 104 (not shown in the illustration). In the embodiment, the electrosurgical crossing assembly 106 is electrically coupled to the electrosurgical generator 102 via an extension cable, such as a form of multimode extension cable 108. The electrosurgical generator 102 is configured to provide a source of energy, such as radiofrequency (RF) energy to the electrosurgical crossing assembly 106 via the cable 108. In some embodiments, the electrosurgical system 100 includes a ground pad electrode, or indifferent (dispersive) patch electrode 110 electrically coupled to the generator 102 for use with the electrosurgical crossing assembly 106 in a monopolar configuration. In some embodiments, the electrosurgical assembly 106 is implemented in a bipolar configuration without an indifferent patch electrode.

The electrosurgical crossing assembly 106 of the illustrated embodiment includes a delivery component 112 and an electrosurgical device such as a transseptal crossing member 114 that, in embodiments, is configured as an elongate catheter assembly. The delivery component 112 includes an elongated shaft 118 having a shaft distal tip 120. The elongated shaft 118 defines a longitudinally extending axial lumen 122. The transseptal crossing member 114 is adapted to be disposed within the lumen 122 and coupled to the RF energy source, such as the generator 102. In some embodiments, the delivery component 112 can include an elongate sheath, and the transseptal crossing member 114 is disposed within the sheath. In another embodiment, the delivery component 112 can include a dilator/sheath assembly, and the transseptal crossing member 114 is disposed within the dilator/sheath assembly. For instance, the elongated shaft 118 includes a distal tapered portion 124 with an enlargement of cross-sectional area with respect to the shaft distal tip 120. As the distal tapered portion 124 is passed through an aperture (such as an aperture in the tissue) from the shaft distal tip 120, the enlargement of cross-sectional area dilates the aperture. The dilator can be configured as a straight dilator, as illustrated, or a curved dilator. The elongated shaft 118 can be made from various materials including insulative materials such as high-density polyethylene (HDPE).

The transseptal crossing member 114 includes an elongate crossing member shaft 130 with a crossing member proximal portion 132 and a crossing member distal portion 134 having a crossing member distal tip 136. The crossing member distal tip 136 includes a distal tip electrode 140 adapted to deliver the RF energy. In embodiments, the distal tip electrode 140 provides a variety of functions in the electrosurgical system 100 including vaporizing tissue, detecting physiological signals in the cardiac anatomy, and tracking the location of the crossing member distal portion 134 or distal tip 136 such as with a mapping system. The crossing member proximal portion 132 includes an end connector 142 configured to electrically couple to cable 108. In some embodiments, the crossing member shaft 130 is constructed from an electrically conductive material having an insulative outer coating, and the distal tip electrode 140 is exposed. In some embodiment, the electrically conductive material is a flexible, shape memory material such as a nickel titanium alloy or nitinol. In some embodiment, the distal tip electrode 140 is selectively electrically couplable via the multimode extension cable 108 to the RF generator 102 and a mapping system. The transseptal crossing member 114 is configured to conduct RF energy from the proximal portion 132 along the crossing member shaft 130 to the electrode 140 and to conduct detected electrical physiological signals from the electrode 140 along the crossing member shaft 130 to the proximal portion 132. In some embodiments, the electrosurgical assembly 106 also includes a plurality of tracking electrodes (not shown) that are electrically coupled to the mapping system such as via cable 108.

Embodiments of the crossing member 114 include a transseptal RF needle and an RF transseptal guidewire. One example of an RF transseptal needle is available under the trade designation NRG transseptal needle from the present assignee, and an example of an RF transseptal guidewire is available under the trade designation VersaCross from the present assignee. In the illustrated example, the transseptal crossing member 114 is configured as a multifunction conductive transseptal guidewire. For instance, the transseptal guidewire 114 can be used, without exchanges, as a guidewire, a transseptal puncture device, a sensing electrode, and as an exchange rail for delivering therapy sheaths. Such embodiments provide efficiencies to medical procedures as the transseptal crossing member 114 configured as a multifunction guidewire performs multiple functions and reduces the amount of device exchanges in the medical procedure. The transseptal crossing member 114 configured as a multifunction guidewire includes the distal tip 136 extendable from the delivery component shaft distal tip 120 such that the delivery component 112 is retractable from the patient over the transseptal crossing member 114 with the distal tip 136 disposed within the heart. The transseptal crossing member 114 is sufficiently thin and flexible to access the various chambers of the heart. The electrode 140 on the crossing member distal tip 136 is operable to deliver RF energy to puncture the atrial septum from the right atrium, and the distal portion 134 of the crossing member shaft 130 can be advanced through the puncture. Once advanced through the puncture and sufficiently extended from within the delivery component 112, the distal portion 134 is biased to form a coil for anchoring the transseptal crossing member 114 configured as a multifunction guidewire beyond the puncture. The delivery component 112 is retractable from the patient over the transseptal crossing member 114 with the distal tip 136 still disposed within the heart. The transseptal crossing member 114 configured as a multifunction guidewire can also support the installation of therapy devices to a therapy location in the patient's heart, such as tubular members or other catheters and for advancing other devices within the heart.

Among the features of the electrosurgical generator 102, the electrosurgical generator 102 is configured to provide the source of RF energy to the electrosurgical device of the electrosurgical assembly 106 for a puncture operation. The generator 102 is electrically coupled via cables to the electrosurgical assembly 106 and patch electrode 110. During a monopolar punction operation of electrosurgical generator 102, a first electrode, often referred to as the active electrode, is provided with the electrosurgical crossing assembly 106 such as distal tip electrode 140 on the transseptal crossing member 114 while another electrode, such as patch electrode 110, is typically located on the back, buttocks, upper leg, or other suitable anatomical location of the patient during surgery and remote from the active electrode. In such a configuration, the patch electrode 110 is often referred to as a patient return electrode. In embodiments, the generator 102 includes a set of electrical terminals that can electrically couple to devices of the electrosurgical system 100. For instance, the generator 102 includes an active terminal 142 to electrically coupled to an active electrode, such as the distal tip electrode 140, and a return terminal 144 to electrically couple to a return electrode, such as the patch electrode 110 or a return electrode on the electrosurgical crossing assembly. RF energy for a puncture function is provided to the active and return terminals. RF energy for a puncture function in a monopolar mode may be provided at a certain voltage and a continuous current (100% on, or 100% duty cycle). At a power setting of 50 Watts for puncturing (although instantaneous power may be higher), voltage can range from approximately 164 to 400 volts root mean square (RMS). The electrosurgical generator 102 can include a plurality of functions and provide a programmed and custom settings via an interface and be couplable to a suite of electrosurgical tools in addition to the transseptal crossing member 114. In one example, the electrosurgical generator 102 provides RF energy to the active electrode as an alternating current having a frequency in the range of 100 kHz to 10 MHz. Typically, this energy is applied in the form of a continuous sinusoidal puncture signal. In some embodiments, the energy is applied in bursts of pulses. The individual pulses in each burst of a pulsed puncture signal typically each have a duration of 300 milliseconds with an interval between pulses of 700 milliseconds but can vary such as based on parameters of the connected transseptal crossing member 114. The actual pulses are often sinusoidal or square waves and bi-phasic, that is alternating positive and negative amplitudes. Accordingly, electrode 140 is often referred to as the active electrode or puncture electrode.

In the illustrated embodiment, the cable 108 includes a set of proximal connectors 152 that are configured to be coupled to the electrosurgical generator 102 and, in some embodiments, to the mapping system, and distal connectors 156 that are configured to be coupled to the crossing assembly 106. The cable 108, which can include a switch, can be employed to configure the transseptal crossing member 114 in a plurality of settings. For example, the cable 108 can be configured in a puncture setting to electrically couple the electrosurgical generator 102, such as via the active terminal 142, with the transseptal crossing member 114 via connectors 152. In the puncture setting, the electrosurgical generator 102 provides the RF energy to an active electrode on the transseptal crossing member 114 via the cable 108. An electrical circuit of RF energy is formed between the active electrode 140 and the patch electrode 110 through the patient, which is used to puncture tissue at the active electrode. The cable 108 can also be configured in a setting to electrically couple the electrosurgical assembly 106 with the mapping system. For example, the electrode 140 on the transseptal crossing member 114 can provides detected physiological signals to the mapping system. Additionally, the cable 108 can be configured to electrically coupled the electrosurgical assembly 106 with the mapping system to receive signals from the electrosurgical assembly 106 to track the location of the distal end portion 134 of the transseptal crossing member 114.

In one embodiment, the electrosurgical generator 102 can program and select particular controls, or ranges of controls, based on the particular configuration of the electrosurgical crossing assembly 106. The electrosurgical crossing assembly 106 of the embodiment includes a memory device 109 (non-transitory memory) storing a set of associated parameters 111. The electrosurgical generator 102 is configured to read the parameters 111 to program the controls to be suited for the associated electrosurgical crossing assembly 106. The memory device 109 can store the parameters 111 in various memory segments having lookup tables or other data structures to provide data to be loaded into a memory device in the electrosurgical generator 102 and read by a controller of the electrosurgical generator 102 to affect operation. Example parameters 111 can include model number of the electrosurgical crossing assembly 106, acceptable power levels signals applied to the puncture assembly 106, whether the crossing assembly 106 is configured for single use or multiple uses, whether the crossing assembly is used in a monopolar mode or a bipolar mode, whether the crossing assembly has additional sensors, as well as other parameters. In some embodiments, the electrosurgical generator 102 can be programmed to write to memory segments on the memory device 109 as well as read the memory device 109.

In an anticipated use of the system 100 in an electrophysiology procedure, the electrosurgical assembly 106 is coupled to the RF generator 102 via cable 108. If the electrosurgical assembly 106 is to be configured in a monopolar mode, the patch electrode 110 is coupled to the patient. The RF generator 102 can be set to a puncture mode, such as an energy output of approximately 50 watts. In some examples, femoral access is obtained via a conventional percutaneous needle, and the transseptal crossing member 114 is inserted into the vasculature and advanced to the superior vena cava. The shaft distal tip 120 of the delivery component 112 is advanced over the proximal portion 132 of the crossing member 114, and the distal tapered portion 124 of the delivery component shaft 118 is advanced over the transseptal crossing member 114 to the superior vena cava. Under visualization, the distal tapered portion 124 is moved from the superior vena cava to the right atrial septum and then to the fossa ovalis of the heart. The location of the assembly 106 is confirmed at the fossa ovalis. For example, the delivery component distal tip 120 is confirmed at the fossa ovalis, such as via visualization, and the electrode 140 of the transseptal crossing member 114 is advanced from the delivery component distal tip 120. The transseptal crossing member 114 is energized to puncture the fossa ovalis. In one example, the exposed electrode 140 of the transseptal guidewire 114 is extended a few millimeters from the delivery component distal tip 120 to tent the heart tissue, and the transseptal crossing member 114, in some examples, can be locked in position with respect to the delivery component 112. Forward pressure is applied to the electrosurgical device 106 and the transseptal crossing member 114 is actuated to apply the RF energy to the electrode 140 and puncture the fossa ovalis. The RF energy vaporizes the cardiac tissue and creates an aperture in the fossa ovalis. The transseptal crossing member 114, in examples, is extended through the aperture in the fossa ovalis. In some procedures, the transseptal crossing member is extended into the pulmonary vein, through the ostium, and anchored.

In embodiments, the transseptal crossing member 114 configured as a multifunction guidewire is extended longitudinally for several millimeters prior to the distal portion 134 curving to assume a J-tip or pigtail shape and deflecting away from the atrial septum. In the embodiment of the delivery component 112 configured as the dilator/sheath assembly, the distal tapered portion 124 is advanced into the puncture site to expand the aperture in the fossa ovalis. The delivery component 112 can be retracted from the patient over the transseptal crossing member 114 configured as a guidewire, and transseptal crossing member 114 can provide support the installation of tubular members or other catheters and for advancing other devices within the heart through the enlarged or expanded aperture in the fossa ovalis.

A benefit of using RF energy to puncture tissue is that the electrosurgical device, such as an electrosurgical blade or the distal tip 136 of the transseptal crossing member 114, is generally inert and atraumatic to the patient until a clinician is ready to perform a crossing. The electrosurgical device will only cut when an RF energy signal is provided from the electrosurgical generator. In some embodiments and as used for illustration in this disclosure, an energy signal is configured to puncture tissue.

Typical electrosurgical generators when activated to provide an RF energy signal will deliver an RF energy signal to the electrosurgical device for a brief period of time. In one example, a clinician can specify an amount of time the energy signal will be applied to the electrosurgical device. Once a clinician activates the electrosurgical generator to apply the energy signal, the electrosurgical device will receive the energy signal for the specified amount of time, barring an error condition. For example, a clinician can select an amount of time, such as one second, for the energy signal to be delivered to the electrosurgical crossing assembly 106 upon activation. Once specified, each activation of the energy signal will last one second, and the energy signal will be deactivated or terminated after one second.

Such time-based activation/deactivation of the energy signal has some disadvantages. For example, a puncture of tissue typically takes less than the full duration of the specified amount of activation time. Continued activation of the energy signal after the crossing applies excess RF energy into the blood pool, which increases thermal-withstand demands on electrical insulation adjacent to the electrode and unintended fowling of the electrode tip as surrounding blood is heated or vaporized. Additionally, many clinicians will apply the energy signal while the electrode of the crossing device is still in the delivery component rather than against the tissue in a “running start” strategy. In the running start strategy, the energy signal is applied to the electrode within the delivery component and retracted from the tissue and then the energized electrode is physically advanced to cross the target tissue. The running start strategy risks the live electrode curving back and recrossing the septum from the left atrium or contacting and traumatizing an undesired structure due to the curved tip profile.

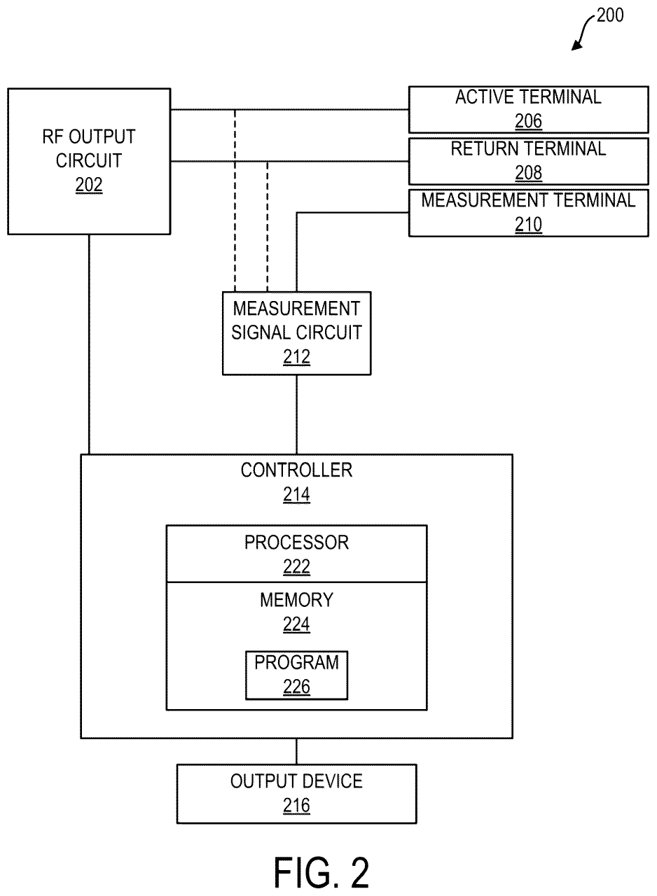

FIG. 2 illustrates an embodiment of an electrosurgical unit, such as an electrosurgical generator 200, which can correspond with electrosurgical generator 102 in system 100. The electrosurgical generator 200 is configured to couple to an electrosurgical device and generate an RF energy signal; the RF energy signal is provided to the electrosurgical device. The RF energy signal is activated, such as by a clinician (or automatically by the generator), to vaporize tissue at a target area of a patient. Also, the electrosurgical generator 200 is configured to generate a measurement signal, provide the measurement signal to the electrosurgical device, and receive a response to the measurement signal from the electrosurgical device. In one embodiment, the measurement signal is provided to the active electrode via the cable 108. In other embodiments, the measurement signal is provided to a separate device on the electrosurgical device via the cable. The electrosurgical device applies the measurement signal to the target area. A response to the measurement signal is provided to the electrosurgical generator 200 via the cable. The electrosurgical generator 200 monitors the received response to the measurement signal to determine whether tissue has been vaporized and penetrated. In some embodiments, the electrosurgical generator 200 monitors the received response to the measurement signal also to determine a tissue type in contact with the electrosurgical device. Based on a determined change in the received response to the measurement signal, the electrosurgical generator 200 infers tissue has been crossed and automatically generates an indication that the tissue has been crossed.

The electrosurgical generator 200 can include several actions to indicate that the tissue has been crossed. In some embodiments, the electrosurgical generator 200 can apply a combination of the actions to indicate the tissue has been crossed. In embodiments, the electrosurgical generator is configured to provide an alert or visualization via an output device such as a display or speaker to notify a clinician that tissue has been crossed. In some embodiments, the electrosurgical generator 200 is configured to determine the tissue being crossed from the response to measurement signal and can provide a visualization such as a text string indicating the tissue being crossed. In embodiments, the electrosurgical generator 200 is configured to modify the RF energy signal or change the parameters of the waveform, such as timing, amplitude, pulse modulation, and control strategy, based on the tissue being crossed or as an indication that the tissue has been crossed. In embodiments, the electrosurgical generator 200 is configured to automatically terminate the energy signal based on a change of the received response to the measurement signal. The automatic termination of the energy signal based on an indication of tissue crossing typically occurs before the time-based deactivation, which reduces the risk of cross back events and reduces RF energy delivered to the patient. Controls are also prepared to terminate the energy signal based on the time-based deactivation if a crossing event is not indicated prior to the time-based deactivation.

The electrosurgical generator 200 includes an RF energy output circuit 202, a plurality of device terminals 204, including an active terminal 206, a return terminal 208, and a measurement signal terminal 210, a measurement signal circuit 212, and a controller 214. In some embodiments, the electrosurgical generator 200 also includes an output device 216, such as a speaker, a display, or both, coupled to the controller 214. In embodiments, the device terminals 204 include connectors to provide a mechanical connection to the electrosurgical generator as well as a signal. For example, the active terminal 206 provides a mechanical and electrical connection with an associated connector of proximal connectors 152 of cable 108. Also, for example, the return terminal 208 provides a mechanical and electrical connection with the patch electrode 110. Still further, for example, the measurement signal terminal 210 provides a mechanical and an electrical or optical (or other signal type) connection with an associated connector of proximal connectors 152 of cable 108. In embodiments, the RF energy output circuit 202 is configured to generate an RF energy signal in response to an input from the controller 214. The RF energy output circuit 202 is electrically coupled to the active terminal 206 and return terminal 208 of the device terminals 204 and provides an energy signal to the active terminal 206. The measurement signal circuit 212 is coupled to the measurement signal terminal 210 and is configured to receive a response to the measurement signal. In some embodiments, the measurement signal circuit also provides the measurement signal. The measurement circuit 212 is electrically coupled to the controller 214 and provides an input representative of the characteristics of the response to the measurement signal to the controller 214. The controller 214 is electrically coupled to the RF energy output circuit 202 and operates the RF energy output circuit 202, such as activates and terminates the energy signal. In one embodiment, a clinician can set a time limit for an amount of time apply the energy signal once the energy signal has been activated. After the time limit has expired, the controller 214 will cause the RF energy output circuit to terminate the energy signal. Typically, tissue is vaporized and a puncture is formed in the tissue very quickly and prior to expiration of the time limit set in the controller 214. In embodiments of the electrosurgical generator 200, the controller 214 causes the RF energy output circuit to terminate the energy signal based on determined changes to the received response to the measurement signal indicating a likelihood that the puncture is formed, which is often prior to the expiration of the time limit.

The RF energy output circuit 202 is configured to generate an RF energy signal in response to an input from the controller 214. The RF energy output circuit 202 can include a power supply to provide a direct current signal and can convert the direct current signal to an alternating current signal. The RF energy output circuit 202 is configured to generate a plurality of voltages, waveforms having various duty cycles, peak voltages, crest factors, frequencies and other suitable parameters. The RF energy output circuit 202 is and provide the energy signal to the active terminal 206 and is electrically coupled to the active terminal 206 and the return terminal 208 to provide the RF energy signal to the active electrode and return electrode, respectively.

The measurement circuit 212 is coupled to the measurement signal terminal 210. The measurement circuit 212 is configured to receive a response to the measurement signal and provide an appropriate signal representative of the response to the measurement signal to the controller 214. In some embodiments, the measurement circuit 212 includes a transducer and circuitry to receive an optical, pressure or other type of signal from the measurement signal terminal 210 and convert to an associated electrical signal suitable for the controller 214. In some embodiments, the response to the measurement signal is an electrical signal and measurement circuit 212 is configured to determine current and voltage measurements or impedance measurements such as magnitude and phase angle received at the measurement signal terminal 210 and present the current and voltage or impedance measurements to the controller 214.

In embodiments, the controller 214 is implemented with any combination of hardware and programming to receive inputs from the measurement circuit 212 and operate the RF energy output circuit 202. In one embodiment, the controller 214 incudes a processor 222 operably coupled to a memory device 224 (a tangible storage medium). The memory device 224 can store processor-executable instructions configured to control the processor 222, such as a program 226. Examples of a memory device 224 can include a non-volatile memory device such as a read only memory (ROM), electronically programmable read only memory (EPROM), flash memory, non-volatile random-access memory (NRAM) or other memory device, and a volatile memory device such as random-access memory (RAM) or other memory device. Memory device 224 can include various combinations of one or both of non-volatile memory devices and volatile memory devices. The processor 222 includes an output port that allows the processor 222 to control the RF energy output circuit 202 according to a programmed scheme.

In other embodiments, the functionalities of controller 214 are at least partially implemented in the form of electronic circuitry. Examples of electronic circuitry include integrated circuits including ASICs and programmable logic devices, such as field programmable gate arrays. A field programmable gate array is a type of integrated circuit that can be programmed or reprogrammed after manufacture and include programable logic blocks and interconnects that are configured to perform various digital functions. The logic blocks can be configured to perform combinational functions or as logic gates. Logic blocks can also include memory elements, such as flip-flops or more complete memory devices including volatile and non-volatile memory aspects that can include look up tables. Functions can be defined via a hardware description language in an electronic design automation tool to create a binary file to configure the electronic circuitry. Those skilled in the art recognize that descriptions of methods, processes, of this disclosure illustrated with the processor 222, memory 224, and program 226 can be implemented in such electronic circuitry.

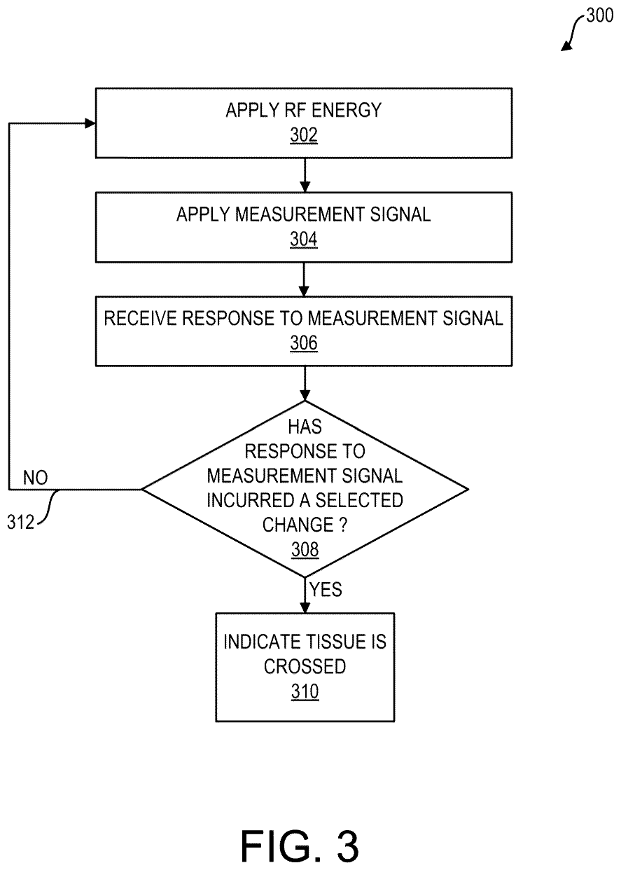

FIG. 3 illustrates an example method 300 that can be implemented in the controller 214, such as via a set of executable instructions, to automatically generate an indication of tissue crossing, such as adjust a waveform delivery via RF energy circuit 202 including terminate the energy signal or provide a notice like an audio signal or display alert such as to output device 216, once a tissue crossing is suspected. In the method, an RF energy signal configured to cross tissue is provided to the active and return terminals 206, 208 of the device terminals 204 at 302. In some embodiments, a measurement signal is provided to the device terminals 204, such as the measurement terminal 210 in some embodiments, at 304. In embodiments, the measurement signal is applied to the target area via an electrosurgical device coupled to the measurement terminal 210. In some embodiments, the measurement signal is applied to the target area concurrently with the RF energy signal. In some embodiments, the measurement signal and RF energy signal are interlaced together and applied to the target area one at a time, via high-speed switching. The electrosurgical device collects a response to the measurement signal, and provides the response to the measurement signal, itself a signal, via cable 108 to the measurement terminal 210. The electrosurgical generator 200 receives the response to the measurement signal at 306. In one embodiment, the measurement signal is provided and the response to the measurement signal is received in an interlaced manner at the measurement terminal 210. A determination is made as to whether the response to the measurement signal has incurred a change at 308. If so, an indication of tissue crossing is generated at 310. If not, the measurement signal and RF energy signal continue to be applied at 312. Such a change may correlate to a predetermined or selected change associated with tissue crossing.

In an example of method 300, an electrosurgical device is coupled to the electrosurgical generator 200 and used to cut into tissue in a target area. In a laparoscopic embodiment, the electrosurgical device will cut through several layers of tissue at a patient's navel, such as skin, muscle, and fat, to reach a cavity in the body. As a clinician applies RF energy at the target area to cut the tissue, the electrosurgical generator receives a response to a measurement signal. In the embodiment, the response to the measurement signal is based on the tissue being cut, i.e., the measurement signal is used to detect the tissue being cut. The response to the measurement signal of skin can include a first reading, the response to the measurement signal of fat can include a second reading, the response to the measurement signal of muscle can include a third reading, and the response to the measurement signal of air, such as in the cavity, can include a fourth reading. A determination is made as to whether the tissue has been crossed based on the response to the measurement signal. For instance, the response to the measurement signal of the first, second, and third readings (those of tissue) indicate the electrosurgical device is still cutting tissue. But a response to the measurement signal changing to the fourth reading, that of air, indicates the electrosurgical device is within the cavity of the body. Thus, a determination is made that tissue has been crossed once the response to the measurement signal changes to the fourth reading from the first through third readings. The method provides an indication of tissue crossing based on the determined change in the response to the measurement signal at 310.

In another example of method 300, such as a transseptal crossing embodiment, the transseptal crossing member is used to puncture the atrial septum to access the left atrium. The transseptal crossing member will cut through the fossa ovalis and access a blood pool in the left atrium. In some embodiments, the transseptal crossing member will start in a blood pool of the right atrium, cut through the fossa ovalis of the atrial septum and access a blood pool in the left atrium. As a clinician applies RF energy at the target area to puncture the fossa ovalis, the electrosurgical generator receives a response to a measurement signal. In the embodiment, the response to the measurement signal is based on the tissue being punctured, i.e., the measurement signal is used to detect the tissue being punctured. The response to the measurement signal of atrial septum tissue can include a first reading, the response to the measurement signal of blood can include a second reading. A determination is made as to whether the tissue has been crossed based on the response to the measurement signal. For instance, the response to the measurement signal of the first reading indicates the electrosurgical device is still within the atrial septum tissue. But a response to the measurement signal changing to the second reading, that of blood, indicates the electrosurgical device is within the left atrium. Thus, a determination is made that tissue has been crossed once the response to the measurement signal changes to the second reading from the first reading. The method provides an indication of tissue crossing based on the determined change in the response to the measurement signal at 310.

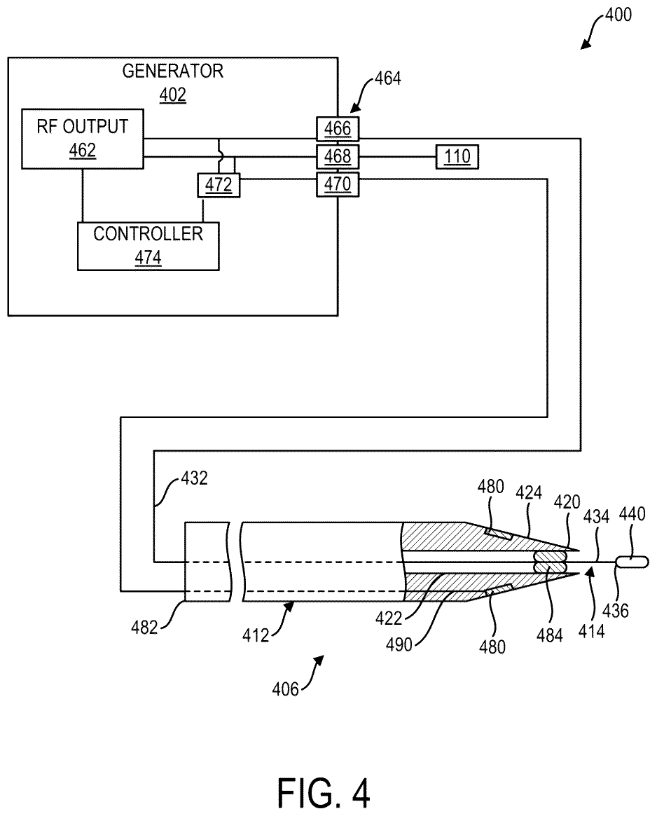

FIG. 4 illustrates an embodiment of an electrosurgical system 400, which corresponds with electrosurgical system 100, configured to measure an electrical characteristic, such as permittivity, of a medium in contact with a crossing device. Permittivity in electromagnetism is the measure of the electric polarizability of a dielectric material and is the ability of the material to store electrical potential energy under the influence of an electric field. A material with high permittivity polarizes more in response to an applied electric field than a material with low permittivity and stores more energy in the material. The embodiment of the electrosurgical system 400 includes an electrosurgical generator 402 and an electrosurgical crossing assembly 406. In the embodiment, the electrosurgical crossing assembly 406 is electrically coupled to the electrosurgical generator 402 via an extension cable, such as a form of multimode extension cable 408.

The electrosurgical generator 402 is an embodiment of the electrosurgical generator 200 of FIG. 2. The electrosurgical generator 402 is configured to couple to an electrosurgical device and generate an RF energy crossing signal; the RF energy crossing signal is provided to the electrosurgical device. The RF energy crossing signal is activated to vaporize tissue at a target area of a patient. Also, the electrosurgical generator 402 is configured to generate a measurement signal, provide the measurement signal to the electrosurgical crossing assembly 406, and receive a response to the measurement signal from the electrosurgical crossing assembly 406. The electrosurgical generator 402 includes an RF energy output circuit 462, a plurality of device terminals 464, including an active terminal 466, a return terminal 468, and a measurement signal terminal 470, a measurement signal circuit 472, and a controller 474.

The electrosurgical crossing assembly 406 of the illustrated embodiment includes a delivery component 412 and an electrosurgical device such as a transseptal crossing member 414 that, in embodiments, is configured as an elongate catheter assembly. The delivery component 412 includes an elongated shaft 418 having a shaft distal tip 420. In the illustration, the distal portion of the delivery component is partially sectioned for clarity. The elongated shaft 418 defines a longitudinally extending axial lumen 422. The transseptal crossing member 414 is adapted to be disposed within the lumen 422 and coupled to the RF energy source, such as the generator 402. In some embodiments, the delivery component 412 can include an elongate sheath, and the transseptal crossing member 414 is disposed within the sheath. In another embodiment, the delivery component 412 can include a dilator/sheath assembly, and the transseptal crossing member 414 is disposed within the dilator/sheath assembly. For instance, the elongated shaft 418 includes a distal tapered portion 424 with an enlargement of cross-sectional area with respect to the shaft distal tip 420. The transseptal crossing member 414 includes an elongate crossing member shaft 430 with a crossing member proximal portion 432 and a crossing member distal portion 434 having a crossing member distal tip 436. The crossing member distal tip 436 includes a distal tip electrode 440 configured to be coupled to the active terminal 466 and adapted to deliver the RF energy. In one embodiment, a liquid saline, can be injected into the lumen 422 and discharged from the distal tip 420 of the shaft 418.

In the illustrated embodiment, the electrosurgical system 400 is also configured to determine an electrical characteristic at the target area, such as permittivity, via a measurement waveform provided to the distal tip electrode 440. Accordingly, the electrosurgical crossing assembly 406 includes a measurement electrode 480 disposed on the distal end 482 of the electrosurgical crossing assembly 406 and spaced apart from the distal tip electrode 440, such as on the crossing member distal portion 434, the distal tapered portion 424, shaft distal tip 420, or another portion. In one embodiment, the measurement electrode 480 is formed from the electrically conductive hypotube of the shaft 418 exposed on the distal tapered portion 424 or shaft distal tip 420. In the illustrated embodiment, the measurement electrode 480 is configured as an electrically conductive ring member exposed on the distal tapered portion 424 of the delivery component 412 and spaced apart from the distal tip electrode 440. Depending on the configuration of the electrosurgical crossing assembly 406, the distal end 482 can include features to electrically isolate the active electrode 440 from the measurement electrode 480 as the active electrode 440 moves relative to the measurement electrode 480. In some embodiments, an electrically insulative guidance member 484, such as a bead disposed around the inner wall of the shaft 418 having a hole therethrough for the transseptal crossing member 414 to pass is included within the lumen 422 to maintain a separation between the active electrode 440 and the measurement electrode 480. The measurement electrode 480 is electrically coupled to an electrical lead 490 that extends along the crossing assembly 406, such as to the shaft proximal end 482. The electrical leads are electrically coupled to connectors suitable for coupling to the measurement terminal 470. The measurement waveform is provided to the distal tip electrode 440 and travels through the target area to the spaced-apart measurement electrode 480 such that an electrical circuit is formed between the active terminal 406 and the measurement terminal 410 of the electrosurgical generator 402.

In one embodiment, the electrosurgical generator 402 is configured to switch between a crossing configuration and a measurement configuration, such as repeatedly switch between a crossing configuration and the measurement configuration. In the crossing configuration, the generator 402 applies an RF energy crossing signal between the active terminal 466 and return terminal 468 to vaporize and cross tissue. In a monopolar mode of the crossing configuration, the return terminal 468 is electrically coupled to a patch electrode 110. In an embodiment of the monopolar mode of the crossing configuration, the measurement terminal 470 is electrically disconnected or isolated from the active terminal 466 within the generator 402. In a bipolar mode of the crossing configuration, however, the electrosurgical crossing assembly 406 can apply the measurement electrode 480 as a return electrode, and the measurement terminal 470 and return terminal 468 can be electrically coupled together such as at the same potential.

In the measurement configuration, the generator 402 applies a measurement signal between the active terminal 466 and measurement terminal 470 to determine electrical characteristics of the measurement signal with the measurement circuit 472. In an embodiment of the measurement configuration, the return terminal 468 is electrically disconnected or isolated from the active terminal 466 within the generator 402. In embodiments, the measurement signal is at a different magnitude and frequency than the RF energy crossing signal. In embodiment, the RF energy output circuit 462 can be configured to generate the RF energy crossing signal having an energy amplitude and energy frequency and also to generate the measurement signal having a measurement amplitude and measurement frequency, wherein at least one of the energy amplitude and energy frequency is different from the measurement amplitude and measurement frequency, respectively.

In embodiments, the electrosurgical generator 402 can be configured in the configurations via data from electrosurgical device, such as data stored on memory device 109 as parameters 111, which can indicate whether the electrosurgical device can be operated in a monopolar mode, bipolar mode, or both, among other operating parameters The generator 402 is configured to connect to an electrosurgical device having an active electrode and a measurement electrode. In the embodiment, the electrosurgical generator 402 is configured to connect to both an electrosurgical device to cross tissue in a monopolar mode, which includes an active electrode at the target site and a return electrode remote from the target site such as a patch electrode 110, and also to an electrosurgical device to cross tissue in a bipolar mode, which includes both an active electrode and a return electrode at the target site. Additionally, the RF output circuit 462 is configured to generate an RF energy crossing signal to cross tissue via an RF energy crossing configuration and to generate a measurement signal via a measurement configuration.

Regardless of whether the electrosurgical crossing assembly 406 is used in a monopolar mode with a patch electrode 110 or a bipolar mode in which the measurement electrode 480 is configured as a return electrode for RF energy to puncture tissue, the electrosurgical generator 402, such as the RF energy output circuit 462, can alternatively generate an RF energy crossing signal and a measurement signal and the electrosurgical generator 402 can correspondingly switch between an RF energy crossing configuration in which the return terminal 468 is electrically coupled to the RF energy output circuit 462 for the RF energy signal and the measurement terminal 470 is electrically coupled to the measurement circuit 472 for the measurement signal. In embodiments, the active terminal 466 and measurement terminal 470 are electrically connected to the measurement circuit 472 to determine electrical characteristics of the measurement signal, such as permittivity.

In the illustrated example, the measurement circuit 472 is applied to measure electrical permittivity between an input, such as the measurement signal provided to the active terminal 466, and an output, such as the response to measurement signal received at the measurement terminal 470. Circuits and devices to measures permittivity between an input and output are known. The measurement circuit 470 provides an indication of the measured permittivity as a signal to the controller 474.

In embodiments in which the generator 402 is coupled to crossing member 414 having an active electrode 440 and a measurement electrode 480, the crossing member 414 operated in a monopolar mode and used with a remote return electrode 110, the generator 402 can be configured in an RF energy crossing configuration to apply an RF energy crossing signal to the active terminal 466 and a measurement configuration to apply a measurement signal to the active terminal 466. For example, the RF energy crossing signal and the measurement signal are each applied to the active electrode 440, such as alternately applied in time.

In the RF energy crossing configuration of the generator 402, when the RF energy crossing signal is applied to the active terminal 466, the return terminal 468 is activated such that an electrical circuit is made between the active terminal 466 coupled to the active electrode 440 and the return terminal 468 coupled to the patch electrode 110. In some embodiments, the measurement terminal 470 is deactivated such that no electrical circuit is formed between the active electrode 440 and the measurement electrode 480 to the measurement circuit 472.

In the measurement configuration of the generator 402, when the measurement signal is applied to the active terminal 466, the measurement terminal is activated such that an electrical circuit is made between the active terminal 466 coupled to the active electrode 440 and the measurement terminal 470 coupled to the measurement electrode 480. In some embodiments, the return terminal 468 is deactivated such that no electrical circuit is formed between the active electrode 440 and the patch electrode 110 to the return terminal 468. The response to the measurement signal is received at the measurement circuit 472, which measurement circuit 472 may also be electrically coupled to the active terminal 468.

In embodiments in which the generator 402 is coupled to an electrosurgical device such as the assembly 406 having an active electrode and a measurement/return electrode operated in a bipolar mode, the generator 402 can be configured in an RF energy crossing configuration to apply an RF energy signal to the active terminal 466 and a measurement configuration to apply a measurement signal to the active electrode. In some embodiments, the patch electrode 110 is not used.

In embodiments of the bipolar mode crossing configuration, the RF output circuit 462 provides an RF energy crossing signal to the active terminal 466, and the output circuit 462 is electrically coupled the return terminal 468 to provide an electrical circuit for the RF energy crossing signal between the active electrode 440 and the measurement electrode 480 (acting as a return electrode at the target site). In some embodiments, the return terminal 468 is not electrically coupled to the measurement circuit 472. In these embodiments of the bipolar mode measurement configuration, the RF output circuit 462 provides a measurement signal to the active terminal 466, and the measurement circuit 472 is electrically coupled to the return terminal 468 to provide an electrical circuit for the measurement signal between the active electrode 440 and the measurement electrode 480 at the target site. In some embodiments, the return terminal 468 is not electrically coupled to the RF output circuit 462. The response to the measurement signal is received at the measurement circuit 472, which measurement circuit 472 may also be electrically coupled to the active terminal 466.

In some embodiments of the bipolar mode, the measurement terminal 470 is not activated and not coupled to the crossing assembly 406. Instead, the crossing assembly 406 is only coupled to the active terminal 466 and the return terminal 468 in some embodiments. The return terminal 468 is coupled to the RF output circuit 462 via the return terminal 468 while a crossing signal is applied to the active terminal 466 from the RF output circuit 462 and to the measurement circuit 472 via the return terminal 468 while the measurement signal is applied to the active terminal 466 via the RF output circuit 462. In other embodiments of the bipolar mode, the return terminal 468 is not activated and not coupled to the crossing assembly 406. Instead, the crossing assembly 406 is only coupled to the active terminal 466 and the measurement terminal 470 in some embodiments. The measurement terminal 470 is coupled to the RF output circuit 462 while a crossing signal is applied to the active terminal 466 from the RF output circuit 462 and to the measurement circuit 472 via the measurement terminal 470 while the measurement signal is applied to the active terminal 466 via the RF output circuit 462.

The electrosurgical system 400 measures the permittivity of medium in which the active electrode 440 and measurement electrode 480 are disposed. For instance, if the active electrode 440 is crossing tissue, the active electrode 440 and measurement electrode 480 are configured to be disposed in (and generate an electric field in) the tissue, and the measurement signal is applied to measure the permittivity of the tissue. For instance, if the active electrode 440 has crossed tissue, the active electrode 440 and measurement electrode 480 are configured to be disposed in (and generate an electric field in) the blood pool of the left atrium, and the measurement signal is applied to measure the permittivity of the blood pool. Based on the measured permittivity, or a determined change in the measured permittivity, the electrosurgical generator 402 is configured to determine whether the electrosurgical crossing assembly has crossed tissue.

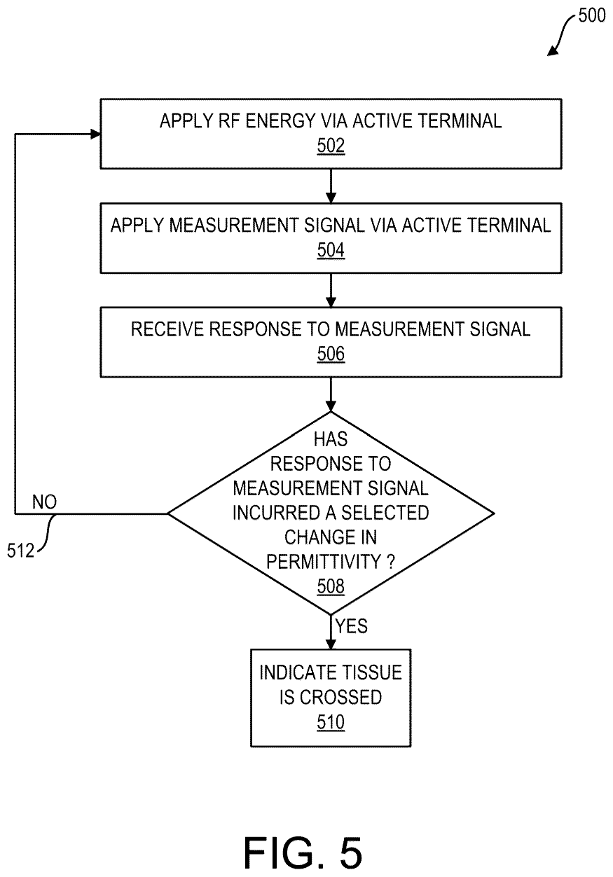

FIG. 5 illustrates an example method 500 that can be implemented in the electrosurgical generator 402, such as via a set of executable instructions, to automatically generate an indication of tissue crossing, such as adjust a waveform delivery via RF energy circuit 462 including terminate the crossing signal or provide a notice like an audio signal or display alert such as to output device, once a tissue crossing is suspected. Method 500 is an embodiment of method 300 of FIG. 3. In the method 500, an RF energy crossing signal configured to cross tissue is provided to the active and return terminals 466, 468 of the device terminals 464 at 502, and the measurement signal is provided to the device terminals 464, such as via the active terminal 466, at 504. In some embodiments, the measurement signal and RF energy signal are interlaced together and applied to the target area one at a time, via high-speed switching. The RF output circuit 402 is configured to generate an RF energy signal via an RF energy configuration and to generate a measurement signal via a measurement configuration. When the RF output circuit 462 is configured to generate a measurement signal via a measurement configuration, the active terminal is electrically coupled to the measurement circuit 472 as an input. The electrosurgical generator 402 receives the response to the measurement signal at 506, such as via the measurement terminal 470 applied as an output to the measurement circuit 472. In one embodiment, the measurement signal is provided and the response to the measurement signal is received in an interlaced manner at the measurement terminal 470. A determination is made as to whether the response to the measurement signal has incurred a change at 508, such as whether the permittivity measurement has changed from a value representative of tissue to a value representative of blood (4190). If so, an indication of tissue crossing is generated at 510. If not, the measurement signal and RF energy signal continue to be applied at 512. In another embodiment, the electrical conductivity values for air, skin, muscle, fat, blood and other bodily media can be applied to also determine crossing via dielectric properties of the media. Such a change may correlate to a predetermined or selected change associated with tissue crossing.

In a laparoscopic example, a determination is made as to whether the response to the measurement signal has incurred a change at 508, such as whether the permittivity measurement has changed from a value representative of tissue to a value representative of air. For instance, a relative permittivity value representative of skin at relevant frequencies is about 1000, a relative permittivity value representative of muscle is about 3650, and a relative permittivity value representative of fat is about is about 5700, whereas a relative permittivity value representative of air is about 1. If so, an indication of tissue crossing is generated at 510. If not, the measurement signal and RF energy signal continue to be applied at 512 to cross tissue. Such a change may correlate to a predetermined or selected change associated with tissue crossing.

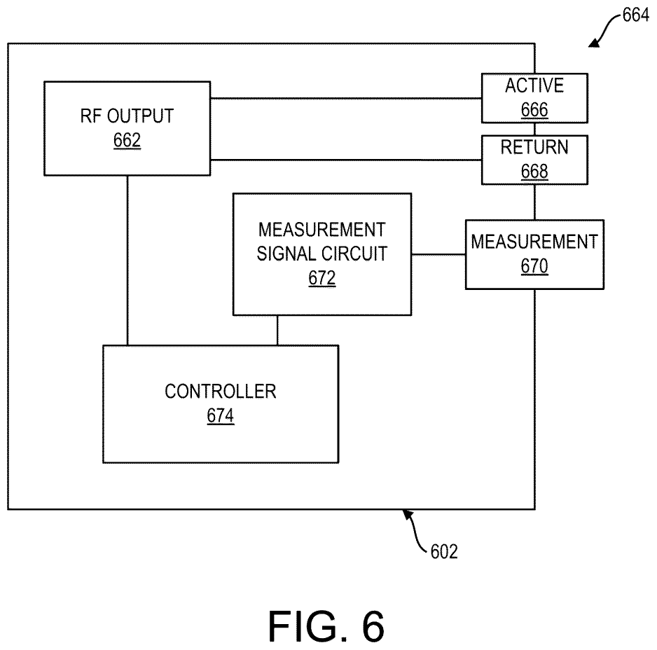

FIG. 6 illustrates an example of an electrosurgical generator 602, which is an embodiment of the electrosurgical generator 200 of FIG. 2. The electrosurgical generator 602 is configured to couple to an electrosurgical device and generate an RF energy crossing signal; the RF energy crossing signal is provided to the electrosurgical device. The RF energy crossing signal is activated, such as by a clinician, to vaporize tissue at a target area of a patient. Also, the electrosurgical generator 602 is configured to generate a measurement signal, provide the measurement signal to the electrosurgical crossing assembly, and receive a response to the measurement signal from the electrosurgical crossing assembly. The electrosurgical generator 602 includes an RF energy output circuit 662, a plurality of device terminals 664, including an active terminal 666, a return terminal 668, and a measurement signal terminal 670, a measurement signal circuit 672, and a controller 674. Unlike embodiments of the electrosurgical generator 402 in which the RF energy output circuit 462 can generate the measurement signal as well as the RF energy crossing signal and the response to the measurement signal is received at the measurement signal circuit 472, the measurement signal circuit 672 in the illustrated embodiment both generates the measurement signal and receives the response to the measurement signal via measurement terminal 670. In some embodiments, the measurement terminal 670 includes a plurality of terminals, such as a first or output terminal to provide a measurement signal and a second or input terminal to receive a response to a measurement signal. In some embodiments, the measurement terminal 670 is a single terminal that serves to provide a measurement signal and receive the response to measurement signal, for instance optical signals that can be multiplexed in the measurement signal circuit 672. In some embodiments, the measurement signal circuit 672 applies a transducer to generate or receive signals, such as an optical transducer or a pressure transducer. In some embodiments, the signals receive at the measurement signal circuit 672 are provided from a transducer at a distal end of the crossing assembly, such as a thermal sensor or a pressure sensor. Other configurations are contemplated. In some embodiments, the active terminal 666 and return terminal 668 are coupled to the RF energy output circuit 662 and do not provide a measurement signal. The RF energy output circuit 662 is configured to generate an RF energy signal to cross or vaporize tissue.

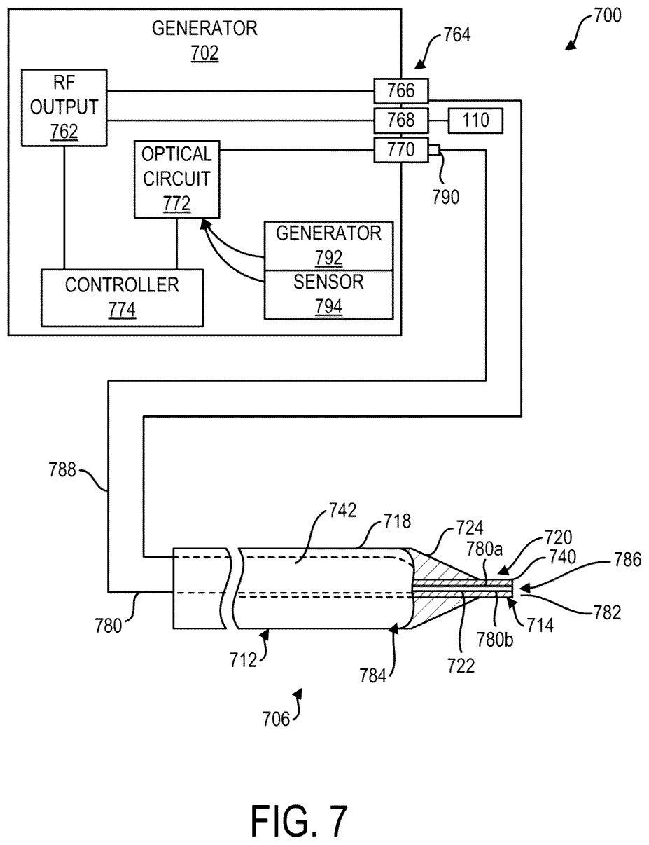

FIG. 7 illustrates an embodiment of an electrosurgical system 700, which corresponds with electrosurgical system 100, configured to measure a characteristic determinable from an optical transducer, such as temperature, pressure, spectroscopy, or time of flight. The embodiment of the electrosurgical system 700 includes an electrosurgical generator 702 and an electrosurgical crossing assembly 706. In the embodiment, the electrosurgical crossing assembly 706 is coupled to the electrosurgical generator 702 to receive an RF energy signal and to transmit and receive an optical signal.

The electrosurgical generator 702 is an embodiment of the electrosurgical generator 200 of FIG. 2 and of generator 600 of FIG. 6. The electrosurgical generator 702 is configured to couple to an electrosurgical device and generate an RF energy signal; the RF energy signal is provided to the electrosurgical device. The RF energy signal is activated, such as by a clinician, to vaporize tissue at a target area of a patient. Also, the electrosurgical generator 702 is configured to generate a measurement signal, provide the measurement signal to the electrosurgical crossing assembly 706, and receive a response to the measurement signal from the electrosurgical crossing assembly 706. The electrosurgical generator 702 includes an RF energy output circuit 762, a plurality of device terminals 764, including an active terminal 766, a return terminal 768, and an optical measurement signal terminal 770, an optical measurement signal circuit 772, and a controller 774.

The electrosurgical crossing assembly 706 of the illustrated embodiment includes a delivery component 712 and an electrosurgical device such as a transseptal crossing member 714 that, in embodiments, is configured as an elongate catheter assembly. The delivery component 712 includes an elongated shaft 718 having a shaft distal tip 720. In the illustration, the distal portion of the delivery component is partially sectioned for clarity. The transseptal crossing member 714 is adapted to be disposed within the delivery component 712 and coupled to the RF energy source, such as the generator 702. In some embodiments, the delivery component 712 can include an elongate sheath, and the transseptal crossing member 714 is disposed within the sheath. In another embodiment, the delivery component 712 can include a dilator/sheath assembly, and the transseptal crossing member 714 is disposed within the dilator/sheath assembly. For instance, the elongated shaft 718 includes a distal tapered portion 724 with an enlargement of cross-sectional area with respect to the shaft distal tip 720. In the illustrated embodiment, the delivery component 712 and crossing member 714 are configured as a unitary piece in which the delivery component does not move with respect to the crossing member 714, and the crossing member 714 includes a tubular distal tip electrode 740 having a longitudinally extending lumen 722 extending from the distal tapered portion 724. Other configurations are contemplated. The delivery component 712 includes an electrical lead 742 electrically coupled to the distal tip electrode 740. The distal tip electrode 740 configured to be coupled to the active terminal 766 such as via the electrical lead 742 and adapted to deliver the RF energy.

In the illustrated embodiment, the electrosurgical system 700 is also configured to determine a characteristic at the target area determinable from an optical transducer—such as temperature, pressure, spectroscopy, or time of flight—via an optical measurement signal provided to the distal end of the crossing assembly 782. Accordingly, the electrosurgical crossing assembly 706 includes an elongate measurement fiber 780, configured from an optical fiber or fiber optical cable, having a distal portion 784 including a distal tip 786 disposed on a distal end 782 of the electrosurgical crossing assembly 706, such as within the lumen 722 at the crossing member distal portion 734, the distal tapered portion 724, shaft distal tip 720, or another portion. The measurement fiber 780 includes a proximal portion 788 coupled to an optical connector 790 configured to be optically connected to the measurement terminal 770. The optical fiber is configured from glass or plastic and can transmit light in the form of an optical signal from one end to another, such as from a proximal end to a distal end or a distal end to a proximal end. In one embodiment, the measurement fiber 780 is coupled to the elongate delivery member shaft 718 and disposed within the lumen 722 of the electrode 740. In another embodiment, the measurement fiber 780 is coupled to the crossing member shaft 730 of the transseptal crossing member 714 and configured to be disposed within the lumen 722. In one embodiment, the measurement fiber 780 includes a single measurement fiber or measurement lead that is configured to both transmit an optical signal and receive a response to the optical signal. In the illustrated embodiment, the measurement fiber 780 includes a set of measurement fibers or measurement leads such as a transmission fiber 780a and a reception fiber 780b.

The optical measurement signal circuit 772 includes electrical circuitry, sensors, or transducers. In one embodiment, the optical measurement signal circuit 772 is configured to generate an optical measurement signal, such as an optical signal based on an electrical signal from the controller 774, with an optical signal generating transducer. In one embodiment, the optical measurement signal circuit 772 receives a signal from the controller 774 and generates a corresponding optical measurement signal having a characteristic, such as various characteristics of amplitude and frequency, with an optical signal generator 792 based on the signal from the controller 774. The optical measurement signal is provided to the optical measurement signal terminal 770 to transmit the optical measurement signal on the measurement fiber 780 such as along the transmission fiber 780a to the distal tip 786 of the measurement fiber 780.

Further, the optical measurement signal circuit 772 is configured to receive an optical response to the measurement signal, such as an optical signal from the reception fiber 780b, and to convert the signal into an appropriate signal from the controller 774. In embodiment, the transmitted optical measurement signal interacts with the target tissue, and the light is received at the distal end 786 of the measurement fiber 780, such as the reception fiber 780b. The optical response to the measurement signal is transmitted from the distal end 786 along the fiber lead to the optical measurement signal terminal 770 optically coupled to the optical measurement signal circuit 772. The optical measurement signal circuit 772 receives the optical response to the measurement signal at an optical sensor 794 and converts the optical response to the measurement signal into a corresponding signal appropriate for use by the controller 774. In one embodiment, the optical sensor 794 is an optical to electrical transducer.

In the illustrated embodiment, the optical signal generator 792 generates the optical measurement signal for transmission along a transmission fiber 780a. The optical measurement signal circuit 772 receives the optical response to the measurement signal from the reception fiber 780b with the optical sensor 794. The generation and reception of optical signals can occur concurrently. For example, the optical measurement terminal 710 include both a transmission component and a reception component for coupling to the measurement fibers 780a, 780b, respectively. In some embodiments, however, the optical signal generator 792 generates the optical measurement signal for transmission along the same optical fiber that receives the optical response to the measurement signal to the optical sensor 794. For example, the optical measurement terminal 770 includes a single optical transmission/reception component for optically coupling to the measurement fiber 780. In one embodiment, the optical signal generator 792 and optical sensor 794 are multiplexed in time such that the optical measurement signal is generated and the optical response to the measurement signal is received at separate times. In embodiments, the RF energy signal does not interfere with optical signals, and the RF energy signal can be applied concurrently with the optical measurement signal.

In one embodiment, the electrosurgical generator 702 is configured to measure time of flight. Time of flight is measurement of the time taken by an object, particle, or wave (such as an acoustic wave or electromagnetic wave) to travel a distance through a medium. This information can be used to measure velocity or path length, or to learn about properties of the medium. In one embodiment, the travel distance is measured as a round trip distance from transducer through the optical fiber, through the medium, reflected off tissue and returned through the medium to the optical fiber and back to the transducer. In another example, the travel distance is the travel from the tip of the optical fiber through the medium, reflected off of tissue and back through the medium to the tip of the optical fiber. Time-of-flight detectors are known and can be implemented with the optical measurement signal circuit 772 and optical transmission and optical reception leads 78a, 780b of the system 700. In a time-of-flight measurement, the optical measurement signal is emitted from the distal end of the transmission fiber 780a, reflected off the tissue in front of the transmission fiber 780a, which reflected waves or particles are received via the reception fiber 780b, and provided to the measurement signal circuit 772. If, for instance, the distal end of the transmission fiber is pressed against or near tissue, such as when the electrosurgical crossing assembly 706 is cutting the tissue, the time of flight of the optical signal will be less than the time of flight of an optical signal that travels through translucent blood and is reflected off a chamber wall, such as when the crossing assembly 706 has crossed tissue and is in a blood pool. Accordingly, in some embodiments, a time-of-flight measurement will increase significantly or spike once tissue has been crossed.

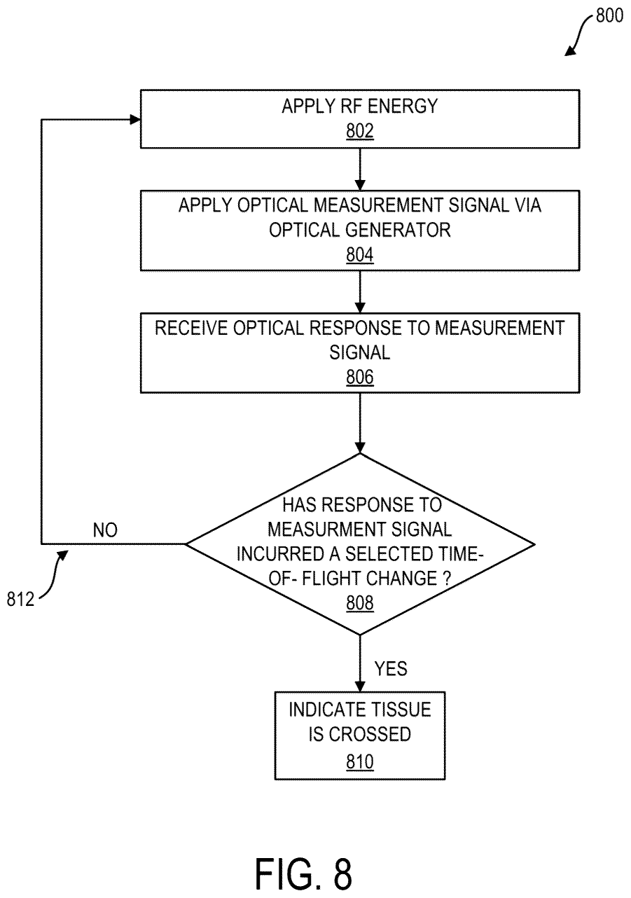

FIG. 8 illustrates an example method 800 that can be implemented in the electrosurgical generator 702, such as via a set of executable instructions, to automatically generate an indication of tissue crossing, such as adjust a waveform delivery via RF energy circuit 762 including terminate the energy signal or provide a notice like an audio signal or display alert such as to output device, once a tissue crossing is suspected. Method 800 is an embodiment of method 300 of FIG. 3. In method 800, an RF energy signal configured to cross tissue is provided to the active terminal 766 and return terminals 768 of the device terminals 764 at 802, and the measurement signal is provided to the device terminals 764, such as a terminal optically coupled to optical signal generator 792 of a time-of-flight measurement circuit, at 804. In some embodiments, the measurement signal is applied to the target area concurrently with the RF energy signal. The electrosurgical generator 702 receives the response to the measurement signal at 806, such as via the measurement terminal 770 applied as an input to the measurement circuit 772, such as the time-of-flight measurement circuit. A determination is made as to whether the response to the measurement signal has incurred a change at 808. In an embodiment in which the electrosurgical generator 702 is configured to measure time of flight, a sharp increase in the time-of-flight measurement can be indicative of a tissue crossing after cutting. If there is a sharp or significant change in time of flight, an indication of tissue crossing is generated at 810. If not, or if the time-of-flight measurement remains within a threshold band, the measurement signal and RF energy signal continue to be applied at 812. Such a change may correlate to a predetermined or selected change associated with tissue crossing.

In another embodiment, the electrosurgical generator 702 of FIG. 7 is configured to quantify tissue composition via diffuse optical imaging (DOI) imaging methods that include diffuse optical spectroscopic imaging (DOSI) and optical spectroscopic tomography (DOT), with optical measurement signal circuit 772 that includes devices or spectrometers such as near-infrared spectroscopic (NIRS) devices, mid-infrared spectroscopic devices, and ultraviolet-visible spectroscopy devices. In one example, the spectroscopic device provides an optical measurement signal via the transmission fiber 780a having a wavelength for non-invasive and ionizing radiation-free assessment of tissue proximate the electrode 740 that is emitted from the distal end of the transmission fiber 780a. Tissue proximate transmission fiber 780a will absorb some of the wavelength spectrum and reflect others of the optical signal, which reflection is received via the distal end of the reception fiber 780b as the response to measurement signal and is provided to the optical measurement signal circuit 772. In a spectroscopic device, the optical measurement signal is emitted an optical signal generator 792, and the optical measurement signal is provided to the optical measurement terminal 770. The reflected optical signal is received at the optical measurement terminal 770 and provided to an optical sensor 794. If, for instance, the distal end of the transmission fiber is pressed against or near tissue, such as when the electrosurgical crossing assembly 706 is cutting the tissue, spectroscopic profile of or measurement of a selected tissue including skin, fat, muscle, blood, is compared against the received response to the optical measurement signal. For instance, the spectroscopic profile of tissue in an atrial septum compares favorably with the response to the optical measurement signal when the crossing assembly 706 is cutting an atrial septum. Also for instance, the spectroscopic profile of a blood pool compares favorably with the response to the optical measurement signal when the crossing assembly 706 has crossed tissue and is in a blood pool. The controller 774 can store a plurality of spectroscopic profiles in memory that can be compared to the response to measurement signal.

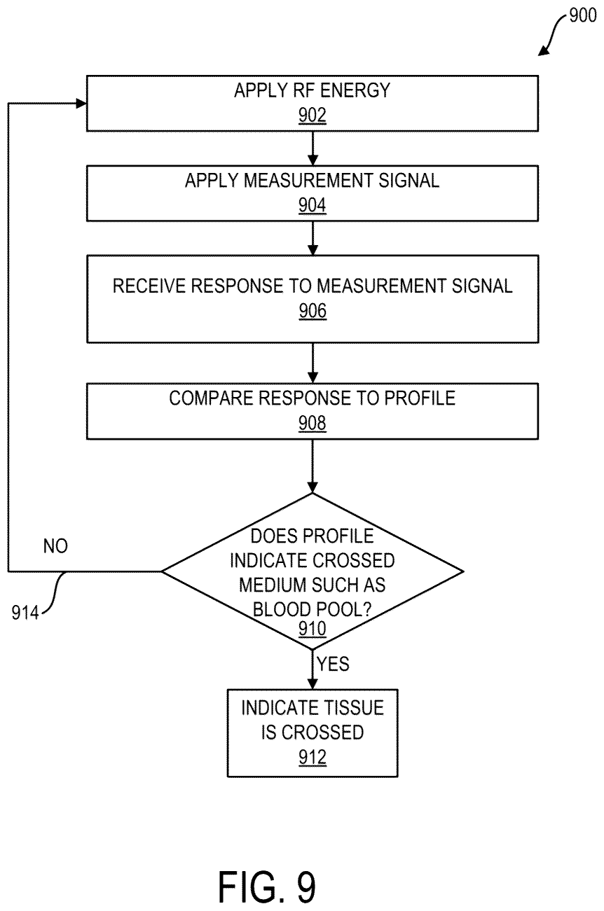

FIG. 9 illustrates an example method 900 that can be implemented in the electrosurgical generator 702, such as via a set of executable instructions, to automatically generate an indication of tissue crossing, such as adjust a waveform delivery via RF energy circuit 762 including terminate the energy signal or provide a notice like an audio signal or display alert such as to output device, once a tissue crossing is suspected. Method 900 is an embodiment of method 300 of FIG. 3. In method 900, an RF energy signal configured to cross tissue is provided to the active and return terminals 766, 768 of the device terminals 764 at 902, and the measurement signal is provided to the device terminals 764, such as a terminal optically coupled to optical signal generator 792 such as a spectrometer circuit, at 904. In some embodiments, the measurement signal is applied to the target area concurrently with the RF energy signal. The electrosurgical generator 702 receives the response to the measurement signal at 906, such as via the measurement terminal 770 applied as an input to the measurement circuit 772 to determine diffuse reflection or absorption of the measurement signal. The response to the measurement signal is compare against a predetermined spectroscopic profile at 908. A determination is made as to whether the response to the measurement signal indicates a certain spectroscopic profile, such as a spectroscopic profile indicating a crossed medium. One example of a crossed medium is of a blood pool. For example, the spectroscopic profile of the atrial tissue, which would be indicated prior to crossing is distinguishable from the spectroscopic profile of a blood pool, which is indicated once the tissue is crossed. In an embodiment in which the electrosurgical generator 702 is configured to quantify tissue composition via DOI, a change in a spectroscopic profile from septum tissue to a blood pool can be indicative of a tissue crossing after cutting. If there is a change in the spectroscopic profile to a blood pool, such as at 910, an indication of tissue crossing is generated at 912. If not, or if detected spectroscopic profile remains that of septum tissue, the measurement signal and RF energy signal continue to be applied at 914.

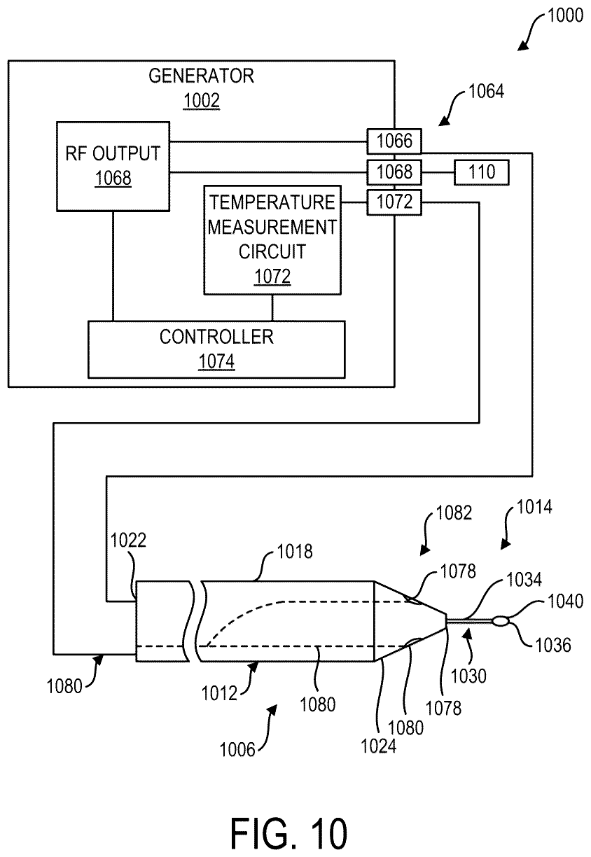

FIG. 10 illustrates an embodiment of an electrosurgical system 1000, which corresponds with electrosurgical system 100, configured to measure a characteristic determinable from a thermal sensor, such as a heat transfer characteristic of tissue. The embodiment of the electrosurgical system 1000 includes an electrosurgical generator 1002 and an electrosurgical crossing assembly 1006. In the embodiment, the electrosurgical crossing assembly 1006 is coupled to the electrosurgical generator 1002 to receive an RF signal and to provide a signal from the temperature sensor.

The electrosurgical generator 1002 is an embodiment of the electrosurgical generator 200 of FIG. 2 and of generator 600 of FIG. 6. The electrosurgical generator 1002 is configured to couple to an electrosurgical device of the electrosurgical crossing assembly 1006 and generate an RF energy signal; the RF energy signal is provided to the electrosurgical device of the electrosurgical crossing assembly 1006. The RF energy signal is activated to vaporize tissue at a target area of a patient. Also, the electrosurgical generator 1002 is configured to receive a temperature signal from the electrosurgical crossing assembly 1006. The electrosurgical generator 1002 includes an RF energy output circuit 1062, a plurality of device terminals 1064, including an active terminal 1066, a return terminal 1068, and a temperature signal terminal 1070, a temperature measurement signal circuit 1072, and a controller 1074.

The electrosurgical crossing assembly 1006 of the illustrated embodiment includes a delivery component 1012 and an electrosurgical device such as a transseptal crossing member 1014 that, in embodiments, is configured as an elongate catheter assembly. The delivery component 1012 includes an elongated shaft 1018 having a shaft distal tip 1020. The elongated shaft 1018 defines a longitudinally extending axial lumen 1022. The transseptal crossing member 1014 is adapted to be disposed within the lumen 1022 and coupled to the RF energy source, such as the generator 1002. In some embodiments, the delivery component 1012 can include an elongate sheath, and the transseptal crossing member 1014 is disposed within the sheath. In another embodiment, the delivery component 1012 can include a dilator/sheath assembly, and the transseptal crossing member 1014 is disposed within the dilator/sheath assembly. For instance, the elongated shaft 1018 includes a distal tapered portion 1024 with an enlargement of cross-sectional area with respect to the shaft distal tip 1020. The transseptal crossing member 1014 includes an elongate crossing member shaft 1030 with a crossing member proximal portion 1032 and a crossing member distal portion 1034 having a crossing member distal tip 1036. The crossing member distal tip 1036 includes a distal tip electrode 1040 configured to be coupled to the active terminal 1066 and adapted to deliver the RF energy.

In the illustrated embodiment, the electrosurgical system 1000 is also configured to determine a characteristic at the target area determinable from a thermal sensor 1078—such as heat transfer characteristics—such as via a thermistor at the distal end of the crossing assembly 1082. In embodiments, the electrosurgical crossing assembly 1006 includes elongate electrical leads 1080, configured from electrically conductive material, having a distal portion coupled to the thermal sensor 1078, which disposed on the distal end 1082 of the electrosurgical crossing assembly 1006, on the distal tapered portion 1024 as in the illustrated example or on the shaft distal tip 1020 or in the crossing member 1014 such as proximate the electrode 1040. The leads 1080 includes a proximal portion coupled to a connector configured to be electrically connected to the measurement terminal 1070. In one embodiment, the lead 1080 is coupled to the elongate delivery member shaft 1018 within the lumen. In another embodiment, the leads 1080 are coupled to the crossing member shaft 1030 of the transseptal crossing member 1014 and configured to be disposed within the lumen 1022. The temperature measurement circuit 1072 is coupled to the measurement terminal 1070 and is configured to generate a measurement signal to provide to the thermal sensor, such as a current or voltage, and receive a response signal (such as an electrical signal including a current or voltage signal) from the thermal sensor 1078 and generate an appropriate signal for use by the controller 1074. In some embodiments, the temperature measurement signal is incorporated into the circuitry of the controller.

In one embodiment, the electrosurgical generator 1002 is configured to determine and measure heat transfer characteristics of tissue adjacent to or abutting the thermal sensor. Different types of tissue and fluids include different types of heat transfer characteristics. For example, a heat transfer rate of air is approximately 0 mil/min/kg (such as for a laparoscopic use), the heat transfer rate of skin is 100 ml/min/kg, of muscle is 37 ml/min/kg, and of fat is 33 mil/min/kg. A variety of techniques exist that enable real-time measurement of heat flow from temperature changes measured by a thermal sensor. The controller 1074 integrating heat flow measurements from the thermal measurement circuit 1072 regarding signals received from the thermal sensor 1078 estimates the tissue in contact with the electrode 1040 to perform various actions. In another embodiment, the controller 1074 can be used to determine temperature trends from at the electrode 1040 based on such characteristics as thermal ramp rates, and tissue thermal properties can be estimated.

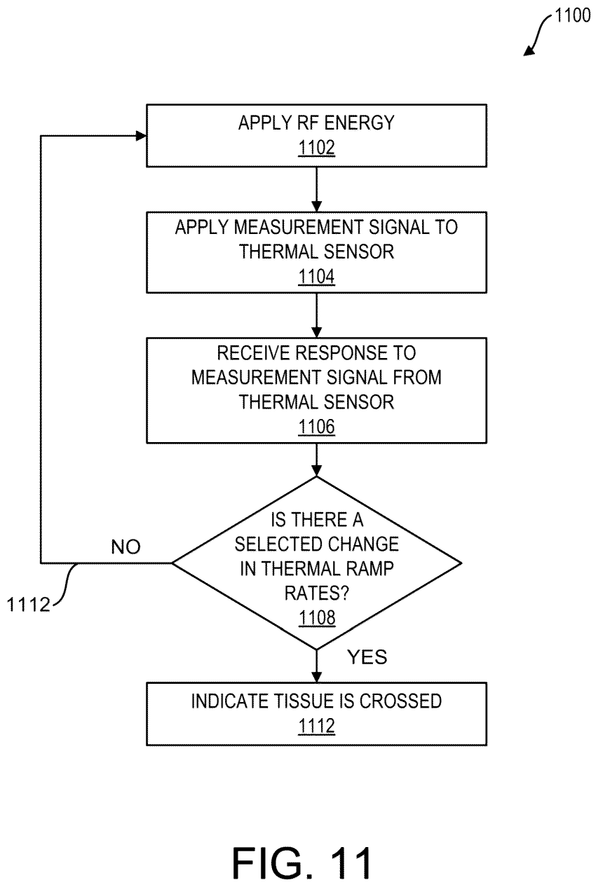

FIG. 11 illustrates an example method 1100 that can be implemented in the electrosurgical generator 1002, such as via a set of executable instructions, to automatically generate an indication of tissue crossing, such as adjust a waveform delivery via RF energy circuit 1062 including terminate the energy signal or provide a notice like an audio signal or display alert such as to output device, once a tissue crossing is suspected. Method 1100 is an embodiment of method 300 of FIG. 3. In method 1100, an RF energy signal configured to cross tissue is provided to the active and return terminals 1066, 1068 of the device terminals 1064 at 1102, and the measurement signal is provided to the device terminals 1064, such as a terminal electrically coupled to thermal signal generator 1092, at 1104. In some embodiments, the measurement signal is applied to the target area via the thermal sensor 1078 concurrently with the RF energy signal. The electrosurgical generator 1002 receives the response to the measurement signal at 1106, such as via the measurement terminal 1068 applied as an input to the measurement circuit 1072. A determination is made as to whether the response to the measurement signal has incurred a change at 1108. In an embodiment in which the electrosurgical generator 1002 is configured to measure temperature trends, such as thermal ramp rates, or rates of change in temperature over time, changes in thermal ramp rates can be indicative of a tissue crossing after cutting. If there is a change in the thermal ramp rate corresponding with a tissue crossing, an indication of tissue crossing is generated at 1110. If not, or if the thermal ramp rate remains within a threshold band, the measurement signal and RF energy signal continue to be applied at 1112. For example, if the heat transfer rate drops to 0 in a laparoscopic use, the device has crossed tissue and is in air. The RF energy signal can be terminated.

In another embodiment, the electrosurgical generator 1002 of FIG. 10 is configured to quantify temperature measurements and temperature measurement over time during a procedure as a heat transfer profile, or a heat-flow profile. If, for instance, the thermal sensor 1078 is pressed against or near tissue, such as when the electrosurgical crossing assembly 1006 is cutting the tissue, a temperature reading or characteristics of the temperature reading such as changes in temperature over time, can be applied to determine a heat transfer profile. Heat transfer profiles, such as signatures of a selected tissue including skin, fat, muscle, blood, are compared against the determined heat transfer profile determined from the received response to the temperature measurement signal. The controller 1074 can store a plurality of heat transfer profiles of procedures in memory that can be compared to the response to measurement signal.

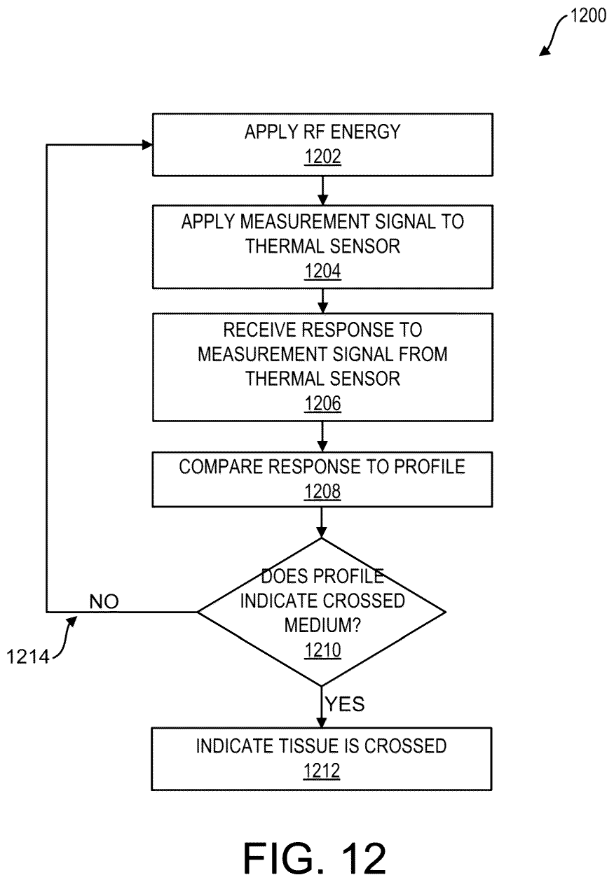

FIG. 12 illustrates an example method 1200 that can be implemented in the electrosurgical generator 1002, such as via a set of executable instructions, to automatically generate an indication of tissue crossing, such as adjust a waveform delivery via RF energy circuit 1062 including terminate the energy signal or provide a notice like an audio signal or display alert such as to output device, once a tissue crossing is suspected. Method 1200 is an embodiment of method 300 of FIG. 3. In method 1100, an RF energy signal configured to cross tissue is provided to the active and return terminals 1066, 1068 of the device terminals 1064 at 1202, and the measurement signal is provided to the device terminals 1064, such as a terminal electrically coupled to thermal signal generator 1092, at 1204. In some embodiments, the measurement signal is applied to the target area via the thermal sensor 1078 concurrently with the RF energy signal. The electrosurgical generator 1002 receives the response to the measurement signal at 1206, such as via the measurement terminal 1068 applied as an input to the measurement circuit 1072 to determine heat transfer characteristics of the tissue adjacent to or abutting against the thermal sensor 1078. The response to the measurement signal is compare against a predetermined heat transfer profile at 1208. A determination is made as to whether the response to the measurement signal indicates a certain heat transfer profile at 1210, such as a heat transfer profile of a blood pool or a heat transfer profile of the atrial septum. Various heat transfer profiles can be stored in a memory device and made available to the controller for comparison at 1208. In an embodiment in which the electrosurgical generator 702 is configured to quantify tissue composition via heat transfer profile, a change in a heat transfer profile from septum tissue to a blood pool can be indicative of a tissue crossing after cutting. If there is a change in the heat transfer profile to a blood pool, an indication of tissue crossing is generated at 1212. If not, or if detected heat transfer profile remains that of septum tissue, the measurement signal and RF energy signal continue to be applied at 1214.

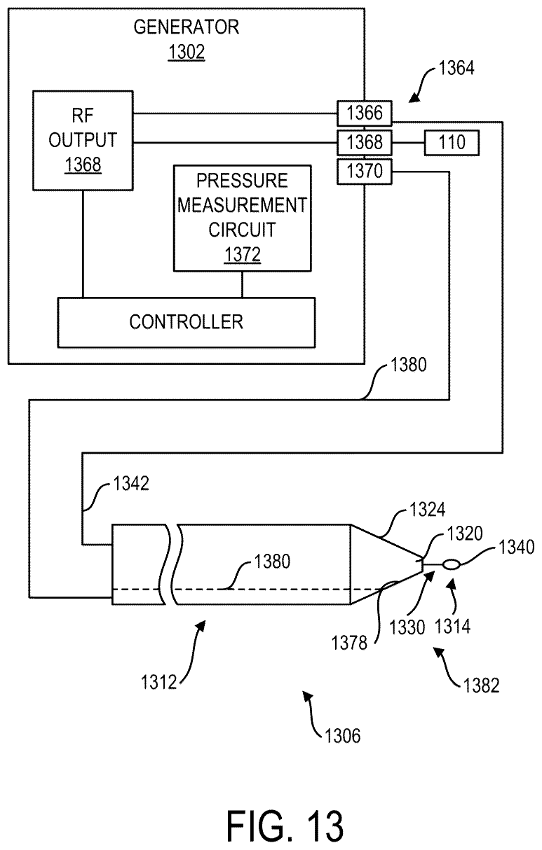

FIG. 13 illustrates an embodiment of an electrosurgical system 1300, which corresponds with electrosurgical system 100, configured to measure a characteristic determinable from a pressure sensor, such as a pressure characteristic of tissue or the target area. The embodiment of the electrosurgical system 1300 includes an electrosurgical generator 1302 and an electrosurgical crossing assembly 1306. In the embodiment, the electrosurgical crossing assembly 1306 is coupled to the electrosurgical generator 1302 to receive an RF signal and to provide a signal from the pressure sensor.

The electrosurgical generator 1302 is an embodiment of the electrosurgical generator 200 of FIG. 2 and of generator 600 of FIG. 6. The electrosurgical generator 1302 is configured to couple to an electrosurgical device of the electrosurgical crossing assembly 1306 and generate an RF energy signal; the RF energy signal is provided to the electrosurgical device of the electrosurgical crossing assembly 1306. The RF energy signal is activated to vaporize tissue at a target area of a patient. Also, the electrosurgical generator 1302 is configured to receive a pressure signal from the electrosurgical crossing assembly 1306. The electrosurgical generator 1302 includes an RF energy output circuit 1362, a plurality of device terminals 1364, including an active terminal 1366, a return terminal 1368, and a pressure signal terminal 1370, a pressure measurement signal circuit 1372, and a controller 1374.

The electrosurgical crossing assembly 1306 of the illustrated embodiment includes a delivery component 1312 and an electrosurgical device such as a transseptal crossing member 1314 that, in embodiments, is configured as an elongate catheter assembly. The delivery component 1312 includes an elongated shaft having a shaft distal tip 1320. The elongated shaft defines a longitudinally extending axial lumen. The transseptal crossing member 1314 is adapted to be disposed within the lumen and coupled to the RF energy source, such as the generator 1302. In some embodiments, the delivery component 1312 can include an elongate sheath, and the transseptal crossing member 1314 is disposed within the sheath. In another embodiment, the delivery component 1312 can include a dilator/sheath assembly, and the transseptal crossing member 1314 is disposed within the dilator/sheath assembly. For instance, the elongated shaft includes a distal tapered portion 1324 with an enlargement of cross-sectional area with respect to the shaft distal tip 1320. The transseptal crossing member 1314 includes an elongate crossing member shaft 1330 with a crossing member proximal portion and a crossing member distal portion having a crossing member distal tip. The crossing member distal tip includes a distal tip electrode 1340 configured to be electrically coupled to the active terminal 1366 via RF energy lead 1342 and adapted to deliver the RF energy.

In the illustrated embodiment, the electrosurgical system 1300 is also configured to determine a characteristic at the target area determinable from a pressure sensor 1378—such as pressure characteristics—such as via a pressure transducer at the distal end of the crossing assembly 1382. In one embodiment, the electrosurgical crossing assembly 1306 includes an elongate electrical lead 1380, configured from electrically conductive material, having a distal portion coupled to the pressure sensor 1078, which disposed on the distal end 1382 of the electrosurgical crossing assembly 1306, on the distal tapered portion 1324 as in the illustrated example or proximate the shaft distal tip 1320 or in the crossing member 1314 such as proximate the electrode 1340. The lead 1380 includes a proximal portion coupled to a connector configured to be electrically connected to the measurement terminal 1370. The pressure measurement circuit 1372 is coupled to the measurement terminal 1370 and is configured to generate a measurement signal to provide to the pressure sensor, such as a certain current or voltage, and receive a response signal (such as an electrical signal including a current or voltage signal) from the pressure sensor 1378 and generate an appropriate signal for use by the controller 1374. In some embodiments, the pressure measurement signal is incorporated into the circuitry of the controller.

In another embodiment, the pressure sensor includes a fluid extending along the crossing assembly and in fluid communication with the measurement terminal 1370 and the measurement circuit 1372. The measurement circuit includes a transducer in this embodiment to convert the fluid pressure to an electrical signal.

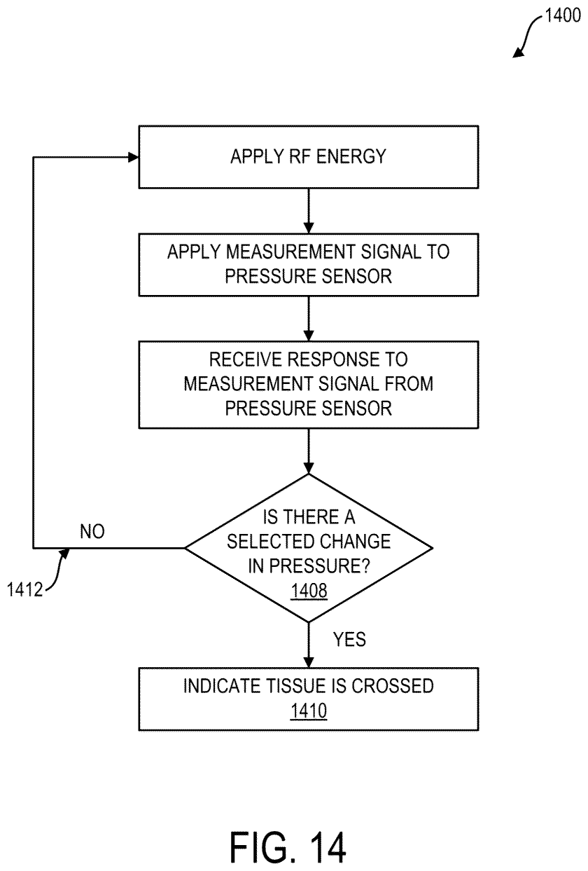

FIG. 14 illustrates an example method 1400 that can be implemented in the electrosurgical generator 1302, such as via a set of executable instructions, to automatically generate an indication of tissue crossing, such as adjust a waveform delivery via RF energy circuit 1362 including terminate the energy signal or provide a notice like an audio signal or display alert such as to output device, once a tissue crossing is suspected. Method 1400 is an embodiment of method 300 of FIG. 3. In method 1400, an RF energy signal configured to cross tissue is provided to the active and return terminals 1366, 1368 of the device terminals 1364 at 1402, and the measurement signal is provided to the device terminals 1364, such as a terminal electrically coupled to pressure sensor circuit 1372, at 1404. In some embodiments, the measurement signal is applied to the target area via the pressure sensor 1378 concurrently with the RF energy signal. The electrosurgical generator 1302 receives the response to the measurement signal at 1406, such as via the measurement terminal 1368 applied as an input to the pressure measurement circuit 1372. A determination is made as to whether the response to the measurement signal has incurred a change at 1408. If there is a change in the pressure corresponding with a tissue crossing, an indication of tissue crossing is generated at 1410. If not, or if the thermal pressure remains within a threshold band, the measurement signal and RF energy signal continue to be applied at 1412. Such a change may correlate to a predetermined or selected change associated with tissue crossing.

For example, in laparoscopic access procedures, entry into the abdominal cavity will result in a large change in pressure that can be detected by pressure sensors integrated into the crossing device. Pressure can also be used to assess locations in the heart due to differences in blood pressure within different heart chambers.

In some embodiments, the memory device 109 can include data related to the parameters 11 can include threshold values of permittivity, time-of-flight, heat transfer, pressure, and other information for method 300 and its embodiments, and can include information particular to the generator used, electrosurgical device of the procedure, or other variables related to the patient or type of procedure. In some examples, the parameters 111 can include many sets of data related to these variables for use with a wide assortment of generators and measurement types. In some examples, the memory devices can be swapped and a memory device with appropriate parameters for the procedure can be selected and included with the crossing assembly.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An electrosurgical unit for use with a crossing member, the electrosurgical unit comprising:
a plurality of device terminals including an active terminal and a measurement terminal, the plurality of device terminals configured to couple to the crossing member; and
a controller configured to:
generate a radiofrequency (RF) energy signal delivery to the active terminal, the RF energy signal configured to cross tissue;
receive a response to a measurement signal at the measurement terminal;
determine whether the crossing member has crossed the tissue via a determined change in the response to the measurement signal; and
generate an indication of tissue crossing based on the determined change in the response to the measurement signal.

2. The electrosurgical unit of claim 1, wherein the determined change in the response to the measurement signal is based on a threshold.

3. The electrosurgical unit of claim 2, wherein the controller is configured to determine a time-of-flight from the response to the measurement signal, and the determined change in the response to the measurement signal is based on a change of the determined time-of-flight with respect to a threshold time-of-flight.

4. The electrosurgical unit of claim 1, wherein the determined change in the response to the measurement signal is based on a comparison to a stored profile.

5. The electrosurgical unit of claim 4, wherein the controller is configured to determine a spectroscopic profile from the response to the measurement signal, and the determined change in the response to the measurement signal is based on a change of the determined spectroscopic profile from a comparison to a stored spectroscopic profile.

6. The electrosurgical unit of claim 1, and further comprising a measurement circuit coupled to the controller and the measurement terminal to receive the response to the measurement signal.

7. The electrosurgical unit of claim 6, wherein the measurement circuit generates the measurement signal.

8. The electrosurgical unit of claim 6, wherein the measurement circuit is an optical measurement circuit having an optical transducer.

9. The electrosurgical unit of claim 8, wherein the optical measurement circuit is configured to generate an optical measurement signal provided to the measurement terminal.

10. The electrosurgical unit of claim 6, wherein the measurement circuit is a thermal measurement circuit configured to process a signal from a thermal sensor.

11. The electrosurgical unit of claim 6, wherein the measurement circuit is a pressure measurement circuit configured to process a signal from a pressure sensor.

12. The electrosurgical unit of claim 11, wherein the pressure measurement circuit includes a pressure transducer.

13. The electrosurgical unit of claim 1, wherein the controller configured to generate the indication of tissue crossing includes the controller configured to terminate the RF energy signal based on the determined change in the response to the measurement signal.

14. The electrosurgical unit of claim 1, wherein the determined change in a response to a measurement signal is based on a determined change in a measurement of electrical permittivity of the tissue.

15. An electrosurgical unit for use with a crossing member, the electrosurgical unit comprising:
a plurality of device terminals including an active terminal and a measurement terminal, the plurality of device terminals configured to couple to the crossing member; and
a controller configured to:
generate a radiofrequency (RF) energy signal delivery to the active terminal, the RF energy signal configured to cross tissue;
receive a response to a measurement signal at the measurement terminal;
determine whether the crossing member has crossed the tissue via a determined change in the response to the measurement signal; and
terminate the RF energy signal based on the determined change in the response to the measurement signal.

16. The electrosurgical unit of claim 15, wherein the determined change in the response to the measurement signal is based on one of a threshold or a comparison to a stored profile.

17. The electrosurgical unit of claim 15, and further comprising a measurement circuit coupled to the controller and the measurement terminal to receive the response to the measurement signal.

18. An electrosurgical tissue crossing system comprising:
a plurality of device terminals including an active terminal, a return terminal, and a measurement terminal;
a crossing member having distal portion including a crossing electrode and a measurement probe; the crossing electrode electrically coupled to the active terminal, the measurement probe operably coupled to the measurement terminal;
a ground pad dispersive electrode electrically coupled to the return terminal; and
a controller configured to:
generate a radiofrequency (RF) energy signal delivery to the active terminal, the RF energy signal configured to cross tissue;
generate a measurement signal, the measurement signal configured to be delivered to the measurement probe;
receive a response to the measurement signal at the measurement terminal, the response from the measurement signal received from the measurement probe;
determine whether the crossing member has crossed the tissue via a determined change in the response to the measurement signal; and
terminate the RF energy signal based on the determined change in the response to the measurement signal.

19. The electrosurgical tissue crossing system of claim 18, wherein the determined change in a response to a measurement signal is based on a determined change in a measurement of electrical permittivity of the tissue.

20. The electrosurgical tissue crossing system of claim 18, wherein the crossing member further includes a delivery component for accessing a heart.
